SEQUENCE LISTING (14 sequences)

SEQ ID NO: 1 - DNA, 2300 bp, Homo sapiens
  misc_feature: cDNA sequence of the variant 1 of the HOXC4 gene (NM_014620.4, NP_055435.2)
ttattgtggtttgtccgttccgagcgctccgcagaacagtcctccctgtaagagcctaaccattgccagggaaacctgccctgggcgctcccttcattagcagtattttttttaaattaatctgattaataattatttttcccccatttaattttttttcctcccaggtggagttgccgaagctgggggcagctggggagggtggggatgggaggggagagacagaagttgagggcatctctctcttccttcccgaccctctggcccccaaggggcaggaggaatgcaggagcaggagttgagcttgggagctgcagatgcctccgcccctcctctctcccaggctcttcctcctgcccccttcttgcaactctccttaattttgtttggcttttggatgattataattatttttatttttgaatttatataaagtatatgtgtgtgtgtgtggagctgagacaggctcggcagcggcacagaatgagggaagacgagaaagagagtgggagagagagaggcagagagggagagagggagagtgacagcagcgctcgcgggggctcaacccccagacctccagaaatgacgtcagaatcatttgcatcccgctgcctctacctgcctggtccagctgggaccctgcctcgccggccgcatggccagagggttggaaattaatgatcatgagctcgtatttgatggactctaactacatcgatccgaaatttcctccatgcgaagaatattcgcaaaatagctacatccctgaacacagtccggaatattacggccggaccagggaatcgggattccagcatcaccaccaggagctgtacccaccaccgcctccgcgccctagctaccctgagcgccagtatagctgcaccagtctccaggggcccggcaattcgcgaggccacgggccggcccaggcgggccaccaccaccccgagaaatcacagtcgctctgcgagccggcgcctctctcaggcgcctccgcctccccgtccccagccccgccagcctgcagccagccagcccccgaccatccctccagcgccgccagcaagcaacccatagtctacccatggatgaaaaaaattcacgttagcacggtgaaccccaattataacggaggggaacccaagcgctcgaggacagcctatacccggcagcaagtcctggaattagagaaagagtttcattacaaccgctacctgacccgaaggagaaggatcgagatcgcccactcgctgtgcctctctgagaggcagatcaaaatctggttccaaaaccgtcgcatgaaatggaagaaggaccaccgactccccaacaccaaagtcaggtcagcacccccggccggcgctgcgcccagcaccctttcggcagctaccccgggtacttctgaagaccactcccagagcgccacgccgccggagcagcaacgggcagaggacattaccaggttataaaacataactcacacccctgcccccaccccatgcccccaccctcccctcacacacaaattgactcttatttatagaatttaatatatatatatatatatatatatataggttcttttctctcttcctctcaccttgtcccttgtcagttccaaacagacaaaacagataaacaaacaagccccctgccctcctctccctcccactgttaaggacccttttaagcatgtgatgttgtcttagcatggtacctgctgggtgtttttttttaaaaggccattttggggggttatttattttttaagaaaaaaagctgcaaaaattatatattgcaaggtgtgatggtctggcttgggtgaatttcaggggaaatgaggaaaagaaaaaaggaaagaaattttaaagccaattctcatccttctcctcctcctccttccccccctctttccttaggccttttgcattgaaaatgcaccaggggaggttagtgagggggaagtcattttaaggagaacaaagctatgaagttcttttgtattattgttgggggggggtgtgggaggagagggggcgaagacagcagacaaagctaaatgcatctggagagcctctcagagctgttcagtttgaggagccaaaagaaaatcaaaatgaactttcagttcagagaggcagtctataggtagaatctctccccacccctatcgtggttattgtgtttttggactgaatttacttgattattgtaaaacttgcaataaagaattttagtgtcgatgtgaaatgccccgtgatcaataataaaccagtggatgtgaattagtttta

SEQ ID NO: 2 - protein, 264 aa, Homo sapiens
  misc_feature: amino acid sequence of the variant 1 of the HOXC4 gene (NM_014620.4, NP_055435.2)
MIMSSYLMDSNYIDPKFPPCEEYSQNSYIPEHSPEYYGRTRESGFQHHHQELYPPPPPRPSYPERQYSCTSLQGPGNSRGHGPAQAGHHHPEKSQSLCEPAPLSGASASPSPAPPACSQPAPDHPSSAASKQPIVYPWMKKIHVSTVNPNYNGGEPKRSRTAYTRQQVLELEKEFHYNRYLTRRRRIEIAHSLCLSERQIKIWFQNRRMKWKKDHRLPNTKVRSAPPAGAAPSTLSAATPGTSEDHSQSATPPEQQRAEDITRL

SEQ ID NO: 3 - DNA, 1689 bp, Homo sapiens
  misc_feature: cDNA sequence of the variant 2 of the HOXC4 gene (NM_153633.2, NP_705897.1)
agaaaaacgacaaagcgagaaaaattattttccactccagaaattaatgatcatgagctcgtatttgatggactctaactacatcgatccgaaatttcctccatgcgaagaatattcgcaaaatagctacatccctgaacacagtccggaatattacggccggaccagggaatcgggattccagcatcaccaccaggagctgtacccaccaccgcctccgcgccctagctaccctgagcgccagtatagctgcaccagtctccaggggcccggcaattcgcgaggccacgggccggcccaggcgggccaccaccaccccgagaaatcacagtcgctctgcgagccggcgcctctctcaggcgcctccgcctccccgtccccagccccgccagcctgcagccagccagcccccgaccatccctccagcgccgccagcaagcaacccatagtctacccatggatgaaaaaaattcacgttagcacggtgaaccccaattataacggaggggaacccaagcgctcgaggacagcctatacccggcagcaagtcctggaattagagaaagagtttcattacaaccgctacctgacccgaaggagaaggatcgagatcgcccactcgctgtgcctctctgagaggcagatcaaaatctggttccaaaaccgtcgcatgaaatggaagaaggaccaccgactccccaacaccaaagtcaggtcagcacccccggccggcgctgcgcccagcaccctttcggcagctaccccgggtacttctgaagaccactcccagagcgccacgccgccggagcagcaacgggcagaggacattaccaggttataaaacataactcacacccctgcccccaccccatgcccccaccctcccctcacacacaaattgactcttatttatagaatttaatatatatatatatatatatatatataggttcttttctctcttcctctcaccttgtcccttgtcagttccaaacagacaaaacagataaacaaacaagccccctgccctcctctccctcccactgttaaggacccttttaagcatgtgatgttgtcttagcatggtacctgctgggtgtttttttttaaaaggccattttggggggttatttattttttaagaaaaaaagctgcaaaaattatatattgcaaggtgtgatggtctggcttgggtgaatttcaggggaaatgaggaaaagaaaaaaggaaagaaattttaaagccaattctcatccttctcctcctcctccttccccccctctttccttaggccttttgcattgaaaatgcaccaggggaggttagtgagggggaagtcattttaaggagaacaaagctatgaagttcttttgtattattgttgggggggggtgtgggaggagagggggcgaagacagcagacaaagctaaatgcatctggagagcctctcagagctgttcagtttgaggagccaaaagaaaatcaaaatgaactttcagttcagagaggcagtctataggtagaatctctccccacccctatcgtggttattgtgtttttggactgaatttacttgattattgtaaaacttgcaataaagaattttagtgtcgatgtgaaatgccccgtgatcaataataaaccagtggatgtgaattagttttaaaaaaaaaaaaaaaaaaaaaaaa

SEQ ID NO: 4 - protein, 264 aa, Homo sapiens; identical to SEQ ID NO: 2
  misc_feature: amino acid sequence of the variant 2 of the HOXC4 gene (NM_153633.2, NP_705897.1)

SEQ ID NO: 5 - DNA, 1681 bp, Homo sapiens
  misc_feature: cDNA sequence of the variant 1 of the HOXC6 gene (NM_004503.3, NP_004494.1)
ttttgtctgtcctggattggagccgtccctataaccatctagttccgagtacaaactggagacagaaataaatattaaagaaatcatagaccgaccaggtaaaggcaaagggatgaattcctacttcactaacccttccttatcctgccacctcgccgggggccaggacgtcctccccaacgtcgccctcaattccaccgcctatgatccagtgaggcatttctcgacctatggagcggccgttgcccagaaccggatctactcgactcccttttattcgccacaggagaatgtcgtgttcagttccagccgggggccgtatgactatggatctaattccttttaccaggagaaagacatgctctcaaactgcagacaaaacaccttaggacataacacacagacctcaatcgctcaggattttagttctgagcagggcaggactgcgccccaggaccagaaagccagtatccagatttacccctggatgcagcgaatgaattcgcacagtggggtcggctacggagcggaccggaggcgcggccgccagatctactcgcggtaccagaccctggaactggagaaggaatttcacttcaatcgctacctaacgcggcgccggcgcatcgagatcgccaacgcgctttgcctgaccgagcgacagatcaaaatctggttccagaaccgccggatgaagtggaaaaaagaatctaatctcacatccactctctcggggggcggcggaggggccaccgccgacagcctgggcggaaaagaggaaaagcgggaagagacagaagaggagaagcagaaagagtgaccaggactgtccctgccacccctctctccctttctccctcgctccccaccaactctcccctaatcacacactctgtatttatcactggcacaattgatgtgttttgattccctaaaacaaaattagggagtcaaacgtggacctgaaagtcagctctggaccccctccctcaccgcacaactctctttcaccacgcgcctcctcctcctcgctcccttgctagctcgttctcggcttgtctacaggcccttttccccgtccaggccttgggggctcggaccctgaactcagactctacagattgccctccaagtgaggacttggctcccccactccttcgacgcccccacccccgccccccgtgcagagagccggctcctgggcctgctggggcctctgctccagggcctcagggcccggcctggcagccggggagggccggaggcccaaggagggcgcgccttggccccacaccaacccccagggcctccccgcagtccctgcctagcccctctgccccagcaaatgcccagcccaggcaaattgtatttaaagaatcctgggggtcattatggcattttacaaactgtgaccgtttctgtgtgaagatttttagctgtatttgtggtctctgtatttatatttatgtttagcaccgtcagtgttcctatccaatttcaaaaaaggaaaaaaaagagggaaaattacaaaaagagagaaaaaaagtgaatgacgtttgtttagccagtaggagaaaataaataaataaataaatcccttcgtgttaccctcctgtataaatccaacctctgggtccgttctcgaatatttaataaaactgatattatttttaaaacttta

SEQ ID NO: 6 - protein, 235 aa, Homo sapiens
  misc_feature: amino acid sequence of the variant 1 of the HOXC6 gene (NM_004503.3, NP_004494.1)
MNSYFTNPSLSCHLAGGQDVLPNVALNSTAYDPVRHFSTYGAAVAQNRIYSTPFYSPQENVVFSSSRGPYDYGSNSFYQEKDMLSNCRQNTLGHNTQTSIAQDFSSEQGRTAPQDQKASIQIYPWMQRMNSHSGVGYGADRRRGRQIYSRYQTLELEKEFHFNRYLTRRRRIEIANALCLTERQIKIWFQNRRMKWKKESNLTSTLSGGGGGATADSLGGKEEKREETEEEKQKE

SEQ ID NO: 7 - DNA, 2072 bp, Homo sapiens
  misc_feature: cDNA sequence of the variant 2 of the HOXC6 gene (NM_153693.3, NP_710160.1)
ttattgtggtttgtccgttccgagcgctccgcagaacagtcctccctgtaagagcctaaccattgccagggaaacctgccctgggcgctcccttcattagcagtattttttttaaattaatctgattaataattatttttcccccatttaattttttttcctcccaggtggagttgccgaagctgggggcagctggggagggtggggatgggaggggagagacagaagttgagggcatctctctcttccttcccgaccctctggcccccaaggggcaggaggaatgcaggagcaggagttgagcttgggagctgcagatgcctccgcccctcctctctcccaggctcttcctcctgcccccttcttgcaactctccttaattttgtttggcttttggatgattataattatttttatttttgaatttatataaagtatatgtgtgtgtgtgtggagctgagacaggctcggcagcggcacagaatgagggaagacgagaaagagagtgggagagagagaggcagagagggagagagggagagtgacagcagcgctcggacgtcctccccaacgtcgccctcaattccaccgcctatgatccagtgaggcatttctcgacctatggagcggccgttgcccagaaccggatctactcgactcccttttattcgccacaggagaatgtcgtgttcagttccagccgggggccgtatgactatggatctaattccttttaccaggagaaagacatgctctcaaactgcagacaaaacaccttaggacataacacacagacctcaatcgctcaggattttagttctgagcagggcaggactgcgccccaggaccagaaagccagtatccagatttacccctggatgcagcgaatgaattcgcacagtggggtcggctacggagcggaccggaggcgcggccgccagatctactcgcggtaccagaccctggaactggagaaggaatttcacttcaatcgctacctaacgcggcgccggcgcatcgagatcgccaacgcgctttgcctgaccgagcgacagatcaaaatctggttccagaaccgccggatgaagtggaaaaaagaatctaatctcacatccactctctcggggggcggcggaggggccaccgccgacagcctgggcggaaaagaggaaaagcgggaagagacagaagaggagaagcagaaagagtgaccaggactgtccctgccacccctctctccctttctccctcgctccccaccaactctcccctaatcacacactctgtatttatcactggcacaattgatgtgttttgattccctaaaacaaaattagggagtcaaacgtggacctgaaagtcagctctggaccccctccctcaccgcacaactctctttcaccacgcgcctcctcctcctcgctcccttgctagctcgttctcggcttgtctacaggcccttttccccgtccaggccttgggggctcggaccctgaactcagactctacagattgccctccaagtgaggacttggctcccccactccttcgacgcccccacccccgccccccgtgcagagagccggctcctgggcctgctggggcctctgctccagggcctcagggcccggcctggcagccggggagggccggaggcccaaggagggcgcgccttggccccacaccaacccccagggcctccccgcagtccctgcctagcccctctgccccagcaaatgcccagcccaggcaaattgtatttaaagaatcctgggggtcattatggcattttacaaactgtgaccgtttctgtgtgaagatttttagctgtatttgtggtctctgtatttatatttatgtttagcaccgtcagtgttcctatccaatttcaaaaaaggaaaaaaaagagggaaaattacaaaaagagagaaaaaaagtgaatgacgtttgtttagccagtaggagaaaataaataaataaataaatcccttcgtgttaccctcctgtataaatccaacctctgggtccgttctcgaatatttaataaaactgatattatttttaaaacttta

SEQ ID NO: 8 - protein, 153 aa, Homo sapiens
  misc_feature: mino acid sequence of the variant 2 of the HOXC6 gene (NM_153693.3, NP_710160.1)
MLSNCRQNTLGHNTQTSIAQDFSSEQGRTAPQDQKASIQIYPWMQRMNSHSGVGYGADRRRGRQIYSRYQTLELEKEFHFNRYLTRRRRIEIANALCLTERQIKIWFQNRRMKWKKESNLTSTLSGGGGGATADSLGGKEEKREETEEEKQKE

SEQ ID NO: 9 - DNA, 2403 bp, Homo sapiens
  misc_feature: cDNA sequence of transcript variant one of the DLX1 gene (NM_178120, NP_835221)
aagctttgaaccgagtttggggagctcagcagcatcatgcttagacttttcaaagagacaaactccattttcttatgaatggaaagtgaaaacccctgttccgcttaaattgggttccttcctgtcctgagaaacatagagacccccaaaagggaagcagaggagagaaagtcccacacccagaccccgcgagaagagatgaccatgaccaccatgccagaaagtctcaacagccccgtgtcgggcaaggcggtgtttatggagtttgggccgcccaaccagcaaatgtctccttctcccatgtcccacgggcactactccatgcactgtttacactcggcgggccattcgcagcccgacggcgcctacagctcagcctcgtccttctcccgaccgctgggctacccctacgtcaactcggtcagcagccacgcatccagcccctacatcagttcggtgcagtcctacccgggcagcgccagcctcgcccagagccgcctggaggacccaggggcggactcggagaagagcacggtggtggaaggcggtgaagtgcgcttcaatggcaagggaaaaaagatccgtaaacccaggacgatttattccagtttgcagttgcaggctttgaaccggaggttccagcaaactcagtacctagctctgccggagagggcggagctcgcggcctctttgggactcacacagactcaggtcaagatctggttccaaaacaagcgatccaagttcaagaagctgatgaagcagggtggggcggctctggagggtagtgcgttggccaacggtcgggccctgtctgctggctccccacccgtgccgcccggctggaaccctaactcttcatccgggaagggctcaggaggaaacgcgggctcctatatccccagctacacatcgtggtacccttcagcgcaccaagaagctatgcagcaaccccaacttatgtgaggttgcccgcccgtctccttcttgtctccccggcccaggtccctcccgcctccaggtccatccatcccgtccggaaaagaaggacccagagggaagaaggaacagtggaggcgggacgccctccatctcctcggagccccgcgaggtccggcccagcaacttcccggcatccgcgctctagcctgaaccctggcctgggccgagcagtggcagcagagagtggcctcggagggaagccactgccacctgagacagcccaagcagcaagataaacccgctccacccgacccgccgaccttcagctttgtgggactatcaggaaaaaacaaaacaaaaacaaaatgtagaaaaagcaaaagctcttttctgtcctgtcagtctcctgtctccttttgctctgtctgtgcgctggtaaagtccaggtcctcatccgtccgctgtcctcattctgcggcctcagcaaaaagccacaaggtctgagcggcccgggtcctgccgggctgaccatctccggatcctgggacactctgcctgaccatctgtgtagctggtgtgggaatctgggggcattggagggagggggttttatttattgagaaatggacttcgcctgaggctgtttgccaattcagggttctgctgggcgcaaggaacgcactgttcaaacgcactgtttactttaagcgcacggggagaaacgaataaggaggacgtggtgatttttaatttatacagtaacttttgtacttctctggtatggagagtttggagccgaatgatttgcattttttacatgtccgacattatttaataaataatttttaaaagaaaagaacgataaatgaagccaacatgattttctcatttcgggaggaactctgttgcttcgcctggacaagaaggaaaatgctgatttcctccttgggtagaaagagggagcgagggcaaatggggagtagagagaaaacaggcgagaacaagcactctaattccagtgggctttaaaataagacaaaatcagctttacaacaatccctagaggctcgaccacagaataatgccagtcaccaccctgaacgcacaatctccagtgcaggatctaatgactgtacatattattgttattattattattgttattattgttgttctgtaaacatgttgcacaagcttagcctttttgcgttctgttgtgtgtggctgtaaaaccccatgctttgtgaaatgagaatcttgacatttttcttgtgaaatttggaaaatgtgatcaattgaaatcaactgtgttttgtgttctctatgtcaaagtttagttttatattgagaatgttaacttattgctttgtatcttgggaaaaaaactttgtaaataagttataaagtttctttgagacagtaaaattatgatttcttgaaaaaaaaaaaaaaaaaaaaaaa

SEQ ID NO: 10 - protein, 255 aa, Homo sapiens
  misc_feature: amino acid sequence of transcript variant one of the DLX1 gene (NM_178120, NP_835221)
MTMTTMPESLNSPVSGKAVFMEFGPPNQQMSPSPMSHGHYSMHCLHSAGHSQPDGAYSSASSFSRPLGYPYVNSVSSHASSPYISSVQSYPGSASLAQSRLEDPGADSEKSTVVEGGEVRFNGKGKKIRKPRTIYSSLQLQALNRRFQQTQYLALPERAELAASLGLTQTQVKIWFQNKRSKFKKLMKQGGAALEGSALANGRALSAGSPPVPPGWNPNSSSGKGSGGNAGSYIPSYTSWYPSAHQEAMQQPQLM

SEQ ID NO: 11 - DNA, 2203 bp, Homo sapiens
  misc_feature: cDNA sequence of transcript variant two of the DLX1 gene (NM_001038493.1, NP_001033582.1)
aagctttgaaccgagtttggggagctcagcagcatcatgcttagacttttcaaagagacaaactccattttcttatgaatggaaagtgaaaacccctgttccgcttaaattgggttccttcctgtcctgagaaacatagagacccccaaaagggaagcagaggagagaaagtcccacacccagaccccgcgagaagagatgaccatgaccaccatgccagaaagtctcaacagccccgtgtcgggcaaggcggtgtttatggagtttgggccgcccaaccagcaaatgtctccttctcccatgtcccacgggcactactccatgcactgtttacactcggcgggccattcgcagcccgacggcgcctacagctcagcctcgtccttctcccgaccgctgggctacccctacgtcaactcggtcagcagccacgcatccagcccctacatcagttcggtgcagtcctacccgggcagcgccagcctcgcccagagccgcctggaggacccaggtcaagatctggttccaaaacaagcgatccaagttcaagaagctgatgaagcagggtggggcggctctggagggtagtgcgttggccaacggtcgggccctgtctgctggctccccacccgtgccgcccggctggaaccctaactcttcatccgggaagggctcaggaggaaacgcgggctcctatatccccagctacacatcgtggtacccttcagcgcaccaagaagctatgcagcaaccccaacttatgtgaggttgcccgcccgtctccttcttgtctccccggcccaggtccctcccgcctccaggtccatccatcccgtccggaaaagaaggacccagagggaagaaggaacagtggaggcgggacgccctccatctcctcggagccccgcgaggtccggcccagcaacttcccggcatccgcgctctagcctgaaccctggcctgggccgagcagtggcagcagagagtggcctcggagggaagccactgccacctgagacagcccaagcagcaagataaacccgctccacccgacccgccgaccttcagctttgtgggactatcaggaaaaaacaaaacaaaaacaaaatgtagaaaaagcaaaagctcttttctgtcctgtcagtctcctgtctccttttgctctgtctgtgcgctggtaaagtccaggtcctcatccgtccgctgtcctcattctgcggcctcagcaaaaagccacaaggtctgagcggcccgggtcctgccgggctgaccatctccggatcctgggacactctgcctgaccatctgtgtagctggtgtgggaatctgggggcattggagggagggggttttatttattgagaaatggacttcgcctgaggctgtttgccaattcagggttctgctgggcgcaaggaacgcactgttcaaacgcactgtttactttaagcgcacggggagaaacgaataaggaggacgtggtgatttttaatttatacagtaacttttgtacttctctggtatggagagtttggagccgaatgatttgcattttttacatgtccgacattatttaataaataatttttaaaagaaaagaacgataaatgaagccaacatgattttctcatttcgggaggaactctgttgcttcgcctggacaagaaggaaaatgctgatttcctccttgggtagaaagagggagcgagggcaaatggggagtagagagaaaacaggcgagaacaagcactctaattccagtgggctttaaaataagacaaaatcagctttacaacaatccctagaggctcgaccacagaataatgccagtcaccaccctgaacgcacaatctccagtgcaggatctaatgactgtacatattattgttattattattattgttattattgttgttctgtaaacatgttgcacaagcttagcctttttgcgttctgttgtgtgtggctgtaaaaccccatgctttgtgaaatgagaatcttgacatttttcttgtgaaatttggaaaatgtgatcaattgaaatcaactgtgttttgtgttctctatgtcaaagtttagttttatattgagaatgttaacttattgctttgtatcttgggaaaaaaactttgtaaataagttataaagtttctttgagacagtaaaattatgatttcttgaaaaaaaaaaaaaaaaaaaaaaa

SEQ ID NO: 12 - protein, 129 aa, Homo sapiens
  misc_feature: amino acid sequence of transcript variant two of the DLX1 gene (NM_001038493.1, NP_001033582.1)
MTMTTMPESLNSPVSGKAVFMEFGPPNQQMSPSPMSHGHYSMHCLHSAGHSQPDGAYSSASSFSRPLGYPYVNSVSSHASSPYISSVQSYPGSASLAQSRLEDPGQDLVPKQAIQVQEADEAGWGGSGG

SEQ ID NO: 13 - DNA, 4510 bp, Homo sapiens
  misc_feature: cDNA sequence of the TDRD1 gene (NM_198795.1, NP_942090.1)
gctgaggccaggagggcgcactggggattggaggcgagggaagtgcagggcgcatcccaggcggcagggctcccagcatcggcagtcgccatcaccgccagaccgcagagacaggttcggatccgcggtcctcttgcctctttccaggcctcgatgagtgttaaatcgccatttaatgtgatgtcaagaaataatttggaagcacctccttgtaagatgacagagccatttaattttgagaaaaatgaaaacaagcttccaccacatgagtctttaagaagtcctggaacacttcctaaccaccctaatttcaggctgaaaagctcagagaatggaaataaaaagaacaattttttgctttgtgagcaaaccaaacaatatttggctagtcaggaagacaattcagtttcttcaaacccgaatggcatcaacggagaagtagttggctccaaaggagacaggaaaaaattgccagcaggaaactcagtgtcaccaccaagtgctgaaagtaattcaccacccaaagaagtgaatattaagcctggaaataatgtacgtcctgcaaaatcaaaaaaactaaacaagttggtcgagaattccttgtccataagtaatccagggctcttcacctccttaggacctcctcttcggtccacaacttgccatcgctgtggcctatttggatcgctgaggtgctctcagtgcaagcagacctactattgctccacagcatgtcaaagaagagactggtctgcacacagcatcgtgtgcaggcctgttcagccaaatttccacaaacttgaaaataaatcatctattgaaacaaaggatgtggaggtaaacaataagagtgactgtccacttggagttactaaggaaatagccatttgggctgagagaataatgttttctgatttgagaagtctacaactcaagaaaaccatggaaataaagggtacggttaccgaattcaaacacccaggggacttctacgtgcagttatattcttcagaagttttagaatacatgaaccaactctctgccagcttaaaagaaacatatgcaaatgtgcatgaaaaagactatattcctgttaagggggaagtttgtattgccaagtacactgttgatcagacctggaacagagcaatcatacaaaacgttgatgtgcagcaaaagaaggcacatgtcttatatattgattatggaaatgaagaaataattccattaaacagaatttaccacctcaacaggaacattgacttgtttcctccttgtgccataaagtgctttgtagccaatgttatcccagcagaagggaattggagcagtgattgtatcaaagctactaaaccactgttaatggagcagtactgctccataaagattgtcgacatcttggaagaggaagtggttacctttgctgtagaagttgagctgccaaattcaggaaaacttttagaccatgtgcttatagaaatgggatatggcttgaaacccagtggacaagattctaagaaggaaaatgcagatcaaagtgatcctgaagatgttggaaaaatgacaactgaaaacaacattgtcgtagacaaaagtgacctaatcccaaaagtgttaactttgaatgtaggtgatgagttttgtggtgtggttgcccacattcaaacaccagaagacttcttttgtcaacaactgcaaagtggccgaaagcttgctgaacttcaggcatcccttagcaagtactgtgatcagttgcctccacgctctgatttttatccagccattggtgatatatgttgtgctcagttctcagaggatgatcagtggtaccgtgcctctgttttggcttacgcttctgaagaatctgtactggtcggatatgtagattatggaaactttgaaatccttagtttgatgagactttgtcccataatcccaaagttgttggaattgccaatgcaagctataaagtgtgtactagcaggagtaaagccatcattaggaatttggactccagaagctatttgtctcatgaaaaaacttgtacagaacaaaataatcacagtgaaagtggtggacaagttggaaaacagttccctggtggagcttattgataaatccgagacgcctcatgtcagtgttagcaaagttctcctagatgcaggctttgctgtgggagaacagagtatggtgacagataaacccagtgacgtgaaagaaaccagtgttcccttgggtgtggaaggaaaagtaaatccattggagtggacatgggttgaacttggtgttgaccaaacagtagatgttgtggtctgtgtgatatatagtcctggagaattttattgccatgtgcttaaagaggatgctttaaagaaactcaatgatttgaacaagtcattagcagaacactgccagcagaagttacctaatggtttcaaggcagagataggacaaccttgttgtgctttttttgcaggtgatggtagttggtatcgtgctttagtcaaggaaatcttaccaaatggacatgttaaagtacattttgtggattatggaaacatcgaagaagttactgcagatgaactccgaatgatatcatcaacatttttaaaccttccctttcagggaatacggtgccagttagcagatatacagtctagaaacaaacattggtctgaagaagccataacaagattccagatgtgtgttgctgggataaaattgcaagccagagtggttgaagtcactgaaaatgggataggagttgaactcaccgatctctccacttgttatcccagaataattagtgatgttctgattgatgaacatctggttttaaaatctgcttcaccacataaagacttaccaaatgacagacttgttaataaacatgagcttcaagttcatgtacagggacttcaagctacctcttcagctgagcaatggaagacgatagaattgccagtggataaaactatacaagcaaatgtattagaaatcataagcccaaacttgttttatgctctaccaaaagggatgccagaaaatcaggaaaagctgtgcatgttgacagctgaattattagaatactgcaatgctccgaaaagtcgaccaccctatagaccaagaattggagacgcatgctgtgccaaatacacaagtgatgatttttggtatcgtgcagttgttctggggacatcagacactgatgtggaagtgctctatgcagactatggaaacattgaaaccctgcctctttgcagagtgcaaccaatcacctctagccacctggcgcttcctttccaaattattagatgttcacttgaaggattaatggaattgaatggaagctcttctcaattaataataatgctattaaaaaatttcatgttgaatcagaatgtaatgctttctgtgaaaggaattacaaagaatgtccatacagtgtcagttgagaaatgttctgagaatgggactgtcgatgtagctgataagctagtgacatttggtctggcaaaaaacatcacacctcaaaggcagagtgctttaaatacagaaaagatgtataggatgaattgctgctgcacagagttacagaaacaagttgaaaaacatgaacatattcttctcttcctcttaaacaattcaaccaatcaaaataaatttattgaaatgaaaaaactgttaaaaaaaacagcatctcttggaggtaaacccttatgagacaggaaacagcaaaggctagctttaggagagaaagtacagcacctggtgtttttatttatgagaaccttttctttgtccactttctctgtaatgaccttctatccctccgtttttgcctgcctgccattctcctattaggttggtggtttttattttcctctaagttccttccaccaaataaatattacgtaaaaaattcataccaaatcaatgagaatactggcaaggaatacatagggactttctgctatatatgtaactttttattacttaaaggtaccgaaggaaggccaggtgcagtggctcacgcccagcactttgggaggctgaggtgggaggatcccttgaggccaggagttcaaggttacagtgagctatgatagtgccactgcactccagcctgggtgacagattttgtcttaaaaaaaaaaaaaaaaaagttgatatgagttttattttctgtccgtttgaaatattttgtaatattccctgcattctctgtcgtctgcctcttccacataatgtcctttgctttcatgtttgttatcttctttttctgttcactcagaggtcatcaatttctttctctccgtccttaattggattatttttcttttggcctttgggcacagagtctgacctctggaccactctaactggagaaggaactttatgttccctctcctgctgtgtccacaaccttagaaatctgtagctagatttttgttgttatagatagaatttactgtttctgaaacccaaatacagttatcagtttaaggtt

SEQ ID NO: 14 - protein, 1189 aa, Homo sapiens
  misc_feature: amino acid sequence of the TDRD1 gene (NM_198795.1, NP_942090.1)
MSVKSPFNVMSRNNLEAPPCKMTEPFNFEKNENKLPPHESLRSPGTLPNHPNFRLKSSENGNKKNNFLLCEQTKQYLASQEDNSVSSNPNGINGEVVGSKGDRKKLPAGNSVSPPSAESNSPPKEVNIKPGNNVRPAKSKKLNKLVENSLSISNPGLFTSLGPPLRSTTCHRCGLFGSLRCSQCKQTYYCSTACQRRDWSAHSIVCRPVQPNFHKLENKSSIETKDVEVNNKSDCPLGVTKEIAIWAERIMFSDLRSLQLKKTMEIKGTVTEFKHPGDFYVQLYSSEVLEYMNQLSASLKETYANVHEKDYIPVKGEVCIAKYTVDQTWNRAIIQNVDVQQKKAHVLYIDYGNEEIIPLNRIYHLNRNIDLFPPCAIKCFVANVIPAEGNWSSDCIKATKPLLMEQYCSIKIVDILEEEVVTFAVEVELPNSGKLLDHVLIEMGYGLKPSGQDSKKENADQSDPEDVGKMTTENNIVVDKSDLIPKVLTLNVGDEFCGVVAHIQTPEDFFCQQLQSGRKLAELQASLSKYCDQLPPRSDFYPAIGDICCAQFSEDDQWYRASVLAYASEESVLVGYVDYGNFEILSLMRLCPIIPKLLELPMQAIKCVLAGVKPSLGIWTPEAICLMKKLVQNKIITVKVVDKLENSSLVELIDKSETPHVSVSKVLLDAGFAVGEQSMVTDKPSDVKETSVPLGVEGKVNPLEWTWVELGVDQTVDVVVCVIYSPGEFYCHVLKEDALKKLNDLNKSLAEHCQQKLPNGFKAEIGQPCCAFFAGDGSWYRALVKEILPNGHVKVHFVDYGNIEEVTADELRMISSTFLNLPFQGIRCQLADIQSRNKHWSEEAITRFQMCVAGIKLQARVVEVTENGIGVELTDLSTCYPRIISDVLIDEHLVLKSASPHKDLPNDRLVNKHELQVHVQGLQATSSAEQWKTIELPVDKTIQANVLEIISPNLFYALPKGMPENQEKLCMLTAELLEYCNAPKSRPPYRPRIGDACCAKYTSDDFWYRAVVLGTSDTDVEVLYADYGNIETLPLCRVQPITSSHLALPFQIIRCSLEGLMELNGSSSQLIIMLLKNFMLNQNVMLSVKGITKNVHTVSVEKCSENGTVDVADKLVTFGLAKNITPQRQSALNTEKMYRMNCCCTELQKQVEKHEHILLFLLNNSTNQNKFIEMKKLLKKTASLGGKPL

(19) United States
(12) Patent Application Publication
(10) Pub. No.: US 2023/0151427 A1
(43) Pub. Date: May 18, 2023

(54) COMBINATIONS OF MOLECULAR MARKERS WITH IMPROVED SENSITIVITY AND SPECIFICITY FOR PREDICTING AND TREATING PROSTATE CANCER

(71) Applicant: MDxHealth Research B.V., Nijmegen (NL)

(72) Inventors: Franciscus Petrus Smit, Nijmegen (NL); Daphne Hessels, Malden (NL); Jacobus A. Schalken, Nijmegen (NL)

(73) Assignee: MDxHealth Research B.V., Nijmegen (NL)

(21) Appl. No.: 17/319,705

(22) Filed: May 13, 2021

Related U.S. Application Data
(63) Continuation of application No. 16/428,777, filed on May 31, 2019, now abandoned.
Application No. 16/428,777 is a continuation of application No. 15/042,949, filed on Feb. 12, 2016, now abandoned.
Application No. 15/042,949 is a continuation-in-part of application No. PCT/EP2014/065899, filed on Jul. 24, 2014.

(30) Foreign Application Priority Data
Aug. 13, 2013 (WO) PCT/EP2013/066889

(51) Int. Cl. C12Q 1/6886; A61N 5/10; A61N 7/00

(52) U.S. Cl. CPC C12Q 1/6886; A61N 5/10; A61N 7/00; C12Q 2600/112; C12Q 2600/158

(57) ABSTRACT

The present invention relates to methods for in vitro establishing, or diagnosing, high grade or low grade prostate cancer in a sample, preferably from a readily obtainable sample such as an urine, a prostatic fluid or ejaculate sample or a processed, or derived sample thereof, originating from human individual suspected of suffering from prostate cancer using expression level analysis of a combination of two, three or four molecular markers for prostate cancer. Based on establishing or diagnosing high grade or low grade prostate cancer in the disclosed methods, the methods further may include administering treatment for prostate cancer to the individual. The present methods provide, using a combination of DLX1 and HOXC6; DLX1, HOXC6 and HOXC4; DLX1, HOXC6 and TDRD1; DLX1, HOXC4 and TDRD1 or DLX1, HOXC6, HOXC4 and TDRD1 expression markers expression markers for in vitro establishing prostate cancer (PrCa) or PrCa_total, preferably Sign_PrCa, and optionally administering treatment if prostate cancer is established in the methods.

### CROSS-REFERENCE TO RELATED PATENT APPLICATIONS

The present application is a continuation of U.S. patent application Ser. No. 16/428,777, filed on May 31, 2019, and published as U.S. Publication No. 2019/0284641, on Sep. 19, 2019, which application is a continuation of U.S. application Ser. No. 15/042,949, filed on Feb. 12, 2016, and published as U.S. Publication No. 2016/0194724, on Jul. 7, 2016, which application is a continuation-in-part of International Patent Application No. PCT/EP2014/065899, filed on Jul. 24, 2014, and published in the English language as WO 2015/022164, on Feb. 19, 2015, which application claims the benefit of priority to International Patent Application No. PCT/EP2013/066889, filed on Aug. 13, 2013. The contents of the foregoing applications are incorporated herein by reference in their entireties.

### SEQUENCE LISTING

This application is being filed electronically via EFS-Web and includes an electronically submitted Sequence Listing in .txt format. The .txt file contains a sequence listing entitled “2019-05-31_170826-00130_Seq_List.txt” created on May 13, 2021 and is 46,716 bytes in size. The Sequence Listing contained in this .txt file is part of the specification and is hereby incorporated by reference herein in its entirety.

### BACKGROUND

The present invention relates to methods for in vitro establishing, or diagnosing, high grade or low grade prostate cancer in a sample, preferably from a readily obtainable sample such as an urine, a prostatic fluid or ejaculate sample or a processed, or derived sample thereof, originating from human individual suspected of suffering from prostate cancer using expression level analysis of a combination of two, three or four molecular markers for prostate cancer. The present invention further relates to the use in expression level analysis of these combined markers for in vitro establishing high grade or low grade prostate cancer and to a kit of parts providing expression analysis of combinations of the present molecular markers for establishing high grade or low grade prostate cancer.

In the Western male population, prostate cancer has become a major public health problem. In many developed countries it is not only the most commonly diagnosed malignancy, but it is the second leading cause of cancer related deaths in males as well. Because the incidence of prostate cancer increases with age, the number of newly diagnosed cases continues to increase as the life expectancy of the general population increases. In the United States, approximately 218,000 men, and in Europe approximately 382,000 men are newly diagnosed with prostate cancer every year.

Epidemiology studies show that prostate cancer is an indolent disease and that more men die with prostate cancer than from it. However, a significant fraction of the tumors behave aggressively and as a result approximately 32,000 American men and approximately 89,000 European men die from this disease on a yearly basis.

The high mortality rate is a consequence of the fact that there are no curative therapeutic options for metastatic prostate cancer. Androgen ablation is the treatment of choice in men with metastatic disease. Initially, 70 to 80% of the patients with advanced disease show response to therapy, but with time the majority of the tumors will become androgen independent. As a result most patients will develop progressive disease.

Since there are no effective therapeutic options for advanced prostate cancer, early detection of this tumor is pivotal and can increase the curative success rate. Although the routine use of serum prostate-specific antigen (PSA) testing has undoubtedly increased prostate cancer detection, one of the main drawbacks of the serum PSA (sPSA) test is the low specificity. Also conditions such as benign prostatic hyperplasia (BPH) and prostatitis can lead to an elevated sPSA level. This results in high negative biopsy rates of 70-80% in the so-called ‘grey area’ of PSA levels 4.0-10.0 ng/ml.

Moreover, PSA-based screening has led to the diagnosis of clinically insignificant prostate tumors, i.e. in the absence of screening, these tumors would not have been diagnosed within the patient's lifetime, which results in over-treatment.

Therefore, (non-invasive) molecular tests, that can accurately identify those men who have early stage, clinically localized prostate cancer and who would gain prolonged survival and quality of life from early radical intervention, are urgently needed. The prime challenge for molecular diagnostics is the identification of clinically significant prostate cancer, i.e. a Gleason Score of >=7 and/or percentage biopsy positive cores >=33% and/or clinical stage >=T2 (Epstein criteria). Furthermore, markers predicting and monitoring the response to treatment are urgently needed. Molecular biomarkers identified in tissues can serve as target for new body fluid based molecular tests. A suitable biomarker preferably fulfils the following criteria:
1) it must be reproducible (intra-en inter-institutional); and
2) it must have an impact on clinical management.
   Further, for diagnostic purposes, it is important that the biomarkers are tested in terms of tissue-specificity and discrimination potential between prostate cancer, normal prostate and BPH. Furthermore, it can be expected that (multiple) biomarker-based assays enhance the sensitivity for cancer detection.

Considering the above, there is an urgent need for molecular prognostic biomarkers capable of predicting the biological behaviour of prostate cancer and outcome.

For the identification of new candidate markers for prostate cancer, it is necessary to study expression patterns in malignant as well as non-malignant prostate tissues, preferably in relation to other medical data.

Recent developments in the field of molecular techniques have provided new tools that enabled the assessment of both genomic alterations and proteomic alterations in samples in a comprehensive and rapid manner. These tools have led to the discovery of many new promising biomarkers for prostate cancer. These biomarkers may be instrumental in the development of new tests that have a high specificity in the diagnosis and prognosis of prostate cancer.

For the molecular diagnosis of prostate cancer, genes that are highly up-regulated in prostate cancer compared to low or normal expression in normal prostate tissue are of special interest. Such genes could enable the detection of one tumor cell in a large background of normal cells, and could thus be applied as a diagnostic marker in prostate cancer detection.

In the search for PCa specific biomarkers, two promising candidates have been identified: Prostate CAncer gene 3 (PCA3) and TMPRSS2-ERG gene fusions. These biomarkers can be measured using a non-invasive urine test. The PCA3 gene is highly over-expressed in prostate tumors, and has diagnostic value to predict biopsy outcome, but its prognostic value is limited. The Progensa® PCA3 test is a FDA-approved molecular diagnostic test that is available to urologists.

Gene fusions in which ETS family members are mostly fused to androgen-regulated genes, particularly TIVIPRSS2, are PCa-specific molecular events. TMPRSS2-ERG gene fusions are present in approximately 50% of PCa patients. The prognostic value of this gene fusion is still unclear. Consequently, the urgent need for more accurate prognostic biomarkers for PCa persists.

In the art, there is a continuing need for assays providing establishment, or diagnosis, of all prostate cancers with maximal sensitivity, specificity, positive predictive value (PPV) and negative predictive value (NPV). All prostate cancers include both low-grade (Gleason Score >7) and high-grade (Gleason Score >=7) prostate cancers and will be further referred to as PrCa_total.

Furthermore, there is a continuing need for assays for the prediction, or prognosis, of clinical significant prostate cancer, i.e. a Gleason Score of >=7 and/or a percentage biopsy positive cores >=33% and/or a clinical stage >=T2 (Epstein criteria) with maximal sensitivity, specificity, positive predictive value (PPV) and negative predictive value (NPV). These clinically significant prostate cancers will be further referred to as Sign-PrCa.

### SUMMARY

The present invention utilizes a two, three or four gene based model for improved diagnosis of PrCa_total and/or Sign-PrCa for meeting the above indicated needs of the art.

Sensitivity relates to the assay's ability to identify positive results. In the present context, sensitivity indicates the proportion of individuals suffering from prostate cancer testing positive for PrCa_total or Sign-PrCa.

Specificity relates to the ability of the test to identify negative results. In the present context, specificity is defined as the proportion of individuals not suffering from prostate cancer (based on negative prostate biopsies) testing negative for PrCa_total or Sign-PrCa.

Positive predictive value (PPV) relates to the ability of a test to identify the proportion of positive test results that are true positives. In the present context, PPV is defined as the proportion of individuals with prostate cancer (PrCa_total or Sign-PrCa) testing correctly positive among all positive test results.

Negative predictive value (NPV) relates to the ability of a test to identify the proportion of negative test results that are true negatives. In the present context, NPV is defined as the proportion of individuals without prostate cancer (i.e. negative prostate biopsies) testing correctly negative among all negative test results.

It is an object of the present invention, amongst other objects, to provide an assay for establishing, or diagnosing, PrCa_total or Sign-PrCa in a sample of a human individual suspected to suffer from prostate cancer thereby aiding in the development of an effective clinical strategy to treat prostate cancer.

The above object, amongst other objects, is met by the present invention as outlined in the appended claims providing an assay and means for performing the assay allowing detecting, amongst others, PrCa_total or Sign-PrCa with improved sensitivity, specificity, PPV and NPV.

Specifically, the above object, amongst other objects, is met, according to a first aspect of the present invention, by methods for in vitro establishing prostate cancer (PrCa), or PrCa_total, preferably, preferably Sign_PrCa, in a sample originating from a human individual suspected of or suffering from prostate cancer comprising:
a) determining expression levels of DLX1 and HOXC6, and optionally determining the expression levels of additional biomarkers (e.g., one or more of HOXC4 and TDRD1), and optionally where determining expression levels comprises determining expression levels relative to expression levels of a control (e.g., relative to expression levels of KLK3);
b) establishing up-regulation of the expression levels of DLX1 and HOXC6, and optionally establishing up-regulation of the expression levels of one or more of HOXC4 and TDRD1, as compared to expression levels in a sample originating from an individual not suffering from prostate cancer or as compared to a reference value indicative of a non-disease expression level; and
c) establishing the presence or absence of prostate cancer (PrCa) based on the up-regulation of the expression levels of DLX1 and HOXC6, and optionally based on the up-regulation of one or more of HOXC4 and TDRD1, in said sample.

In the present description, reference is made to human genes, such as one ore more of DLX1, HOXC6, HOXC4, and TDRD1, suitable as biomarkers for prostate cancer by referring to their arbitrarily assigned names. Although the skilled person is readily capable to identify, and use, the present genes as biomarkers based on these names, the appended sequence listing provides both the cDNA sequence and protein sequences of these genes in the public database. Based on the data provided in the tables and figures, the skilled person, without undue experimentation and using standard molecular biology means, will be capable of determining the expression levels of the indicated biomarkers in a sample thereby providing the present methods.

According to a preferred embodiment of this first aspect of the present invention, the present methods, in step (a), further comprise determining the expression level of HOXC4; in step (b) further comprise establishing up-regulation of the expression level of HOXC4 as compared to expression level of HOXC4 in a sample originating from an individual not suffering from prostate cancer or as compared to a reference value indicative of a non-disease expression level; and in step (c), further comprise establishing the presence or absence of prostate cancer (PrCa) based on the up-regulation of the expression levels of DLX1, HOXC6 and HOXC4 in said sample.

According to another preferred embodiment of this first aspect of the present invention, the present methods, in step (a), further comprise determining the expression level of TDRD1; in step (b) further comprise establishing up-regulation of the expression level of TDRD1 as compared to expression level of TDRD1 in a sample originating from an individual not suffering from prostate cancer or as compared to a reference value indicative of a non-disease expression level; and in step (c), further comprise establishing the presence or absence of prostate cancer (PrCa) based on the up-regulation of the expression levels of DLX1, HOXC6 and TDRD1 in said sample.

According to an especially preferred embodiment of this first aspect of the present invention, the present methods, in step (a), further comprise determining the expression level of HOXC4 and TDRD1; in step (b) further comprise establishing up-regulation of the expression level of HOXC4 and TDRD1 as compared to expression level of HOXC4 and TDRD1 in a sample originating from an individual not suffering from prostate cancer or as compared to a reference value indicative of a non-disease expression level; and in step (c), further comprise establishing the presence or absence of prostate cancer (PrCa) based on the up-regulation of the expression levels of DLX1, HOXC6, HOXC4 and TDRD1 in said sample

In the present description, prostate biopsies are considered to be the gold standard for PrCa diagnosis. Negative biopsies indicate normal prostate conditions and will be further referred to as no-PrCa.

In the present description, expression level analysis comprises establishing an increased expression of least two biomarkers selected from the group consisting of HOXC4, HOXC6, DLX1 and TDRD1, or any combination thereof, as compared to expression of these genes in a similar, equivalent, or corresponding sample originating from a human individual not suffering from prostate cancer (no-PrCa). In other words, an increased expression level of a gene or biomarker according to the present invention is a measure of gene expression relative to a non-disease standard.

Suitable combinations of biomarkers according to the present inventon are, amongst others, HOXC4/DLX1; HOXC4/DLX1/TDRD1; HOXC4/DLX1/TDRD1/HOXC6; HOXC4/TDRD1; or DLX1/TDR1.

For example, establishing an increased expression of at least two biomarkers selected from the group consisting of HOXC4, HOXC6, DLX1 and TDRD1, or any combination thereof, as compared to expression of these genes under non-prostate cancer conditions (no-PrCa), allows establishing, or diagnosing prostate cancer (PrCa_total) or significant prostate cancer (Sign-PrCa), thereby providing prognosis and/or prediction of disease survival and an aid to design a clinical treatment protocol.

HOXC4 and HOXC6 are family members of the homeobox superfamily of genes and the HOX subfamily contain members that are transcription factors involved in controlling and coordinating complex functions during development via spatial and temporal expression patterns. In humans, there are 39 classical HOX genes organized into the clusters A, B, C and D.

HOXC4, is one of several homeobox HOXC genes located in a cluster on chromosome 12. Three genes, HOXC4, HOXC5 and HOXC6, share a 5′ non-coding exon. Transcripts may include the shared exon spliced to the gene-specific exons, or they may include only the gene-specific exons. Two alternatively spliced variants that encode the same protein have been described for HOXC4. Transcript variant one represents the longer transcript and includes the shared exon. Transcript variant two includes only gene-specific exons and differs in the 5′UTR compared to variant 1. Within the context of the present invention, HOXC4 expression level determination refers to the expression levels of variants one and two.

Also for HOXC6, alternatively spliced transcript variants encoding different isoforms have been identified. Transcript variant two represents the longer transcript and includes the shared exon. It contains a distinct 5′UTR and lacks an in-frame portion of 5′ coding region compared to variant one. The resulting isoform two has a shorter N-terminus when compared to isoform one. Transcript variant one includes only gene-specific exons and encodes the longer isoform. Within the context of the present invention, HOXC6 expression level determination refers to the expression levels of variants 1 and 2.

TDRD1 is a tudor related gene essential for male germ-cell differentiation. Tudor domains are found in many eukaryotic organisms and have been implicated in protein-protein interactions in which methylated protein substrates bind to these domains. TDRD1 plays a central role during spermatogenesis by participating in the repression transposable elements and prevent their mobilization, which is essential for the germline integrity.

DLX1 belongs to the family of homeodomain transcription factors which are related to the Drosophila distal-less (D11) gene. The family has been related to a number of developmental features and appears to be well preserved across species. Dlx genes are implicated in tangential migration of interneurons from the subpallium to the pallium during vertebrate brain development. It has been suggested that Dlx promotes the migration of interneurons by repressing a set of proteins that are normally expressed in terminally differentiated neurons and act to promote the outgrowth of dendrites and axons.

With respect to DLX1 expression, at least two transcript variants are known. Transcript variant 1 is longer than transcript variant 2 and contains an internal exon in the coding region that results in a frame shift and premature stop codon. Within the context of the present invention, DLX1 expression level determination refers to determination of the expression levels of both transcripts.

According to a preferred embodiment of this first aspect of the present invention, determining expression levels comprises determining mRNA expression levels. In other words, determining expression levels comprises determining transcription levels.

According to another preferred embodiment of this first aspect of the present invention, determining expression levels comprises determining protein levels. In other words, determining expression levels comprises determining translation levels.

According to other particularly preferred embodiments of this first aspect of the present invention, establishing prostate cancer (PrCa) comprises establishing high grade (Gleason score >=7) or low grade (Gleason score <7) prostate cancer and/or establishing prostate cancer (PrCa) comprises establishing a percentage of positive cores of >=33% and/or establishing prostate cancer (PrCa) comprises establishing a clinical stage of >=T2 according to the Epstein criteria.

According to the present invention, the present methods are preferably performed using a sample selected from the group consisting of urine, urine derived, prostatic fluid, prostatic fluid derived, ejaculate and ejaculate derived, an urine, or an urine derived, sample. These samples are the most readily obtainable samples of human bodily derivable samples.

Within the context of the present description, an urine, prostatic fluid or ejaculate derived sample is a sample originating from these bodily fluids, i.e. sample of these fluids further processed, for example, by sedimentation, extraction, precipitation, dilution etc.

According to a second aspect, the present invention relates to the use of a combination of DLX1 and HOXC6; DLX1, HOXC6 and HOXC4; DLX1, HOXC6 and TDRD1; or DLX1, HOXC6, HOXC4 and TDRD1 expression markers expression markers for in vitro establishing prostate cancer (PrCa) or PrCa_total, preferably Sign_PrCa. Other suitable combinations according to this second aspect of the present invention are, amongst others, HOXC4/DLX1; HOXC4/DLX1/TDRD1; HOXC4/TDRD 1; or DLX1/TDR1.

According to a preferred embodiment of this second aspect of the present invention, establishing prostate cancer comprises establishing prostate cancer in a sample selected from the group consisting of urine, urine derived, prostatic fluid, prostatic fluid derived, ejaculate and ejaculate derived.

According to a third aspect, the present invention relates to kits of parts for in vitro establishing prostate cancer in a sample originating from human individual suspected of suffering from prostate cancer comprising:
expression level analysis means for determining the expression levels of a combination of biomarkers selected from the group consisting of HOXC6 and DLX1; HOXC6, DLX1 and HOXC4; HOXC6, DLX1, TDRD1; and HOXC6, DLX1, HOXC4 and TDRD1; and
instructions for use.

Other suitable combinations according this third aspect of the present invention include, amongst others, HOXC4/DLX1; HOXC4/DLX1/TDRD1; HOXC4/TDRD1; or DLX1/TDR1.

In the present kits of parts, the expression level analysis means allow detection and quantification of the gene mRNA expression levels of the indicated gene combinations of HOXC4, HOXC6, DLX1 and TDRD1 using any non-invasive molecular biology technique suitable for the purposes of the invention, such as, for example, expression micro-arrays, quantitative real-time PCR, conventional PCR, NASBA, etc.

Quantitative real-time PCR (qRT-PCR) is preferably used according to the present invention to detect and quantify the present diagnostic and/or prognostic genes. This technique is accurate and it allows quantifying the specific mRNA of the genes of interest.

In a particular advantageous embodiment, the invention provides methods and means for determining whether a sample is to be classified as no prostate cancer no-PrCa (prostate biopsies negative for prostate cancer), PrCa_total (all prostate cancers; low- and high grade) or Sign-PrCa (significant prostate cancer: Gleason Score of >=7 and/or a percentage biopsy positive cores >=33% and/or a clinical stage >=T2).

Considering the heterogeneous nature of prostate tumors, the use of at least two biomarkers appears to be necessary for most, if not all, prostate cancers.

Binary logistic regression analysis can be used to define the best gene panel for the most reliable classification of the patient samples in no prostate cancer, prostate cancer total (diagnosis) or clinical significant prostate cancer (prognosis). The goal of this binary logistic regression analysis is to find the best set of genes so that cases that belong to a particular category of prostate cancer will have a very high calculated probability that they will be allocated to that category.

Using this analysis, combinations of biomarkers selected from the group consisting of HOXC6 and DLX1; HOXC6, DLX1 and HOXC4; HOXC6, DLX1, TDRD1; and HOXC6, DLX1, HOXC4 and TDRD1 can be identified for the diagnosis of prostate cancer (i.e. PrCa_total) and prognosis (i.e. Sign-PrCa) of prostate cancer. Other suitable combinations include, amongst others, HOXC4/DLX1; HOXC4/DLX1/TDRD1; HOXC4/TDRD 1; or DLX1/TDR1.

The foregoing methods further may include performing the foregoing methods to determine the mRNA expression levels of a combination of markers selected from the group consisting of DLX1, HOXC6, HOXC4, and TDRD1 in a biological sample and/or requesting a test providing results of an analysis to determine the mRNA expression levels of a combination of markers selected from the group consisting of DLX1, HOXC6, HOXC4, and TDRD1 in a biological sample. In addition, the foregoing methods may include performing further diagnostic tests and/or requesting results of further diagnostic tests, based on the detected mRNA expression levels of a combination of markers selected from the group consisting of DLX1, HOXC6, HOXC4, and TDRD1 in a biological sample (e.g., based on detecting elevated or reduced expression levels of a combination of markers selected from the group consisting of DLX1, HOXC6, HOXC4, and TDRD1). Further, the foregoing methods may include administering therapy for cancer (e.g., administering a therapy for prostate cancer) based on the detected mRNA expression levels of a combination of markers selected from the group consisting of DLX1, HOXC6, HOXC4, and TDRD1 (e.g., based on detecting elevated or reduced expression levels of a combination of markers selected from the group consisting of DLX1, HOXC6, HOXC4, and TDRD1). The foregoing methods may include administering therapy for cancer (e.g., administering therapy for prostate cancer) to a patient that is exhibiting up-regulation of expression of a combination of markers selected from the group consisting of DLX1, HOXC6, HOXC4, and TDRD1, and optionally that is exhibiting an elevated PSA level (e.g., >4, 5, 6, 7, 8, 9, or 10 ng/ml).

The disclosed methods may be performed utilizing devices, combinations, kits, and/or systems that comprise or utilize components for detecting mRNA expression levels of a combination of markers selected from the group consisting of DLX1, HOXC6, HOXC4, and TDRD1 in a biological sample. In addition, the disclosed methods may be performed utilizing devices, combinations, kits, and/or systems that comprise or utilize components for treating prostate cancer based on the detected mRNA expression levels of a combination of markers selected from the group consisting of DLX1, HOXC6, HOXC4, and TDRD1 in a biological sample. The disclosed devices, combination, kits, and/or systems may comprise or utilize nucleic acid components for detecting mRNA expression levels of a combination of markers selected from the group consisting of DLX1, HOXC6, HOXC4, and TDRD1, and optionally may comprise or utilize nucleic acid components for detecting mRNA expression levels of KLK3. Nucleic acid comprised by or utilized by the disclosed methods, devices, combination, kits, and/or systems may include primers and/or probes for reverse transcribing, amplying, and/or detecting mRNA and/or cDNA of a combination of markers selected from the group consisting of DLX1, HOXC6, HOXC4, and TDRD1, and optionally KLK3 (e.g., where expression level of KLK3 are used as an internal control). Optionally, the devices, combinations, kits, and/or systems may comprise or utilize reagents for isolating nucleic acid from a biological sample, for example, reagents for isolating nucleic acid from a urine sample or for stabilizing nucleic acid in a urine sample. Reagents for isolating nucleic acid from a biological sample may include a nucleic acid stabilization medium comprising or consisting of one or more components selected from the group consisting of salts (e.g., phosphate salts such as sodium phosphate salts, and sulphate salts such as ammonium sulfate salts), acids or buffers (e.g., organic acids such as citric acid), and/or detergents (e.g., dodecyl sulfate salts including lithium salts).

### BRIEF DESCRIPTION OF THE FIGURES

FIG. 1 shows the Receiver Operating Characteristic curve to visualize the diagnostic potential of the individual biomarkers HOXC4, HOXC6, DLX1 and TDRD1 to discriminate PrCa_total from no PrCa in the absence of an arbitrary cut-off value.

FIG. 2 shows the Receiver Operating Characteristic curve to visualize the prognostic potential of the individual biomarkers HOXC4, HOXC6, DLX1 and TDRD1 to discriminate Sign-PrCa from the Rest (i.e. negative biopsy and insignificant PCa) in the absence of an arbitrary cut-off value. Insignificant PrCa is defined as tumors with GS<7, biopsy positive cores <33% and <cT2 stage.

FIG. 3 shows the Receiver Operating Characteristic curve to visualize the diagnostic potential of HOXC6, the combination of HOXC6 with DLX1, the combination of HOXC6 with DLX1 and HOXC4 and the combination of HOXC6 with DLX1 and HOXC4 and TDRD1 to discriminate PrCa_total from no PrCa in the absence of an arbitrary cut-off value.

FIG. 4 shows the Receiver Operating Characteristic curve to visualize the prognostic potential of HOXC6, the combination of HOXC6 with DLX1, the combination of HOXC6 with DLX1 and HOXC4 and the combination of HOXC6 with DLX1 and HOXC4 and TDRD1 to discriminate Sign-PrCa from the Rest (i.e. negative biopsy and insignificant PCa) in the absence of an arbitrary cut-off value.

FIG. 5 shows the Receiver Operating Characteristic curve to visualize the diagnostic potential of the four gene panel (HOXC6, DLX1, HOXC4 and TDRD1) in comparison with mPCA3 levels and serum PSA values to discriminate PrCa_total from no PrCa in the absence of an arbitrary cut-off value.

FIG. 6 shows the Receiver Operating Characteristic curve to visualize the prognostic potential of the four gene panel (HOXC6, DLX1, HOXC4 and TDRD1) in comparison with mPCA3 levels and serum PSA values to discriminate Sign-PrCa from the Rest (i.e. negative biopsy and insignificant PCa) in the absence of an arbitrary cut-off value.

### DETAILED DESCRIPTION

Disclosed are methods, devices, combinations, kits, and systems for diagnosing and treating prostate cancer. The methods, devices, combinations, kits, and systems are described herein using several definitions, as set forth below and throughout the application.

As used in this specification and the claims, the singular forms “a,” “an,” and “the” include plural forms unless the context clearly dictates otherwise. For example, “a component” should be interpreted to mean “one or more components” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The presently disclosed methods, devices, combinations, kits, and/or systems relate to detecting elevated expression of mRNA of the genes DLX1 and/or HOXC6 in a urine sample in order to diagnose and/or prognose an individual, and optionally treat the diagnosed and/or prognosed individual by administering therapy to the individual for treating prostate cancer based on the genetic marker having been identified. Elevated expression of mRNA of the genes DLX1 and/or HOXC6 may be identified relative to an internal control (e.g., expression of mRNA of the gene KLK3) and/or relative to an external control (e.g., expression of mRNA of the genes DLX1 and/or HOXC6 in a patient not having prostate cancer). Expression of mRNA of the genes DLX1 and/or HOXC6 in a urine sample may be normalized relative to expression of mRNA of the gene KLK3 in the urine sample and a HOXC6-DLX1 score may be calculated as disclosed herein (e.g, a HOXC6-DLX1 score of at least about 50, 60, 70, 80, 90, or 100). Further diagnostics may be performed on a sample from the individual based on the HOXC6-DLX1 score (e.g., a Gleason score on a prostate biopsy from the patient) and/or therapy may be administered to the patient based on the HOXC6-DLX1 score.

Optionally, the presently disclosed methods, devices, combinations, kits, and/or systems may relate to detecting elevated expression of mRNA of the genes HOXC4 and/or TDRD1 in a urine sample in order to diagnose and/or prognose an individual, and optionally treat the diagnosed and/or prognosed individual by administering therapy to the individual for treating prostate cancer based on the genetic marker having been identified. Elevated expression of mRNA of the genes HOXC4 and/or TDRD1 may be identified relative to an internal control (e.g., expression of mRNA of the gene KLK3) and/or relative to an external control (e.g., expression of mRNA of the genes HOXC4 and/or TDRD1 in a patient not having prostate cancer). Expression of mRNA of the genes HOXC4 and/or TDRD1 in a urine sample may be normalized relative to expression of mRNA of the gene KLK3 in the urine sample and a HOXC4 and/or TDRD1 score may be calculated as disclosed herein (e.g, a HOXC4 and/or TDRD1 score of at least about 50, 60, 70, 80, 90, or 100). Further diagnostics may be performed on a sample from the individual based on the HOXC4 and/or TDRD1 score (e.g., a Gleason score on a prostate biopsy from the patient) and/or therapy may be administered to the patient based on the HOXC4 and/or TDRD1 score.

As used herein, the term “individual,” which may be used interchangeably with the terms “patient” or “subject,” refers to one who receives medical care, attention or treatment and may encompass a human patient. As used herein, the term “individual” is meant to encompass a person who has a prostate cancer, is suspected of having prostate cancer, or is at risk for developing a prostate cancer. Suitable individuals may include individuals having a serum prostate-specific antigen (sPSA) level of at least about 1, 2, 3, 4, 5, 6, 7, 8, 9, 10, 11, 12, 13, 14, or 15 ng/ml, or individuals having a sPSA level within a range bounded by any of 1, 2, 3, 4, 5, 6, 7, 8, 9, 10, 11, 12, 13, 14, or 15 ng/ml (e.g., individuals having a sPSA level within a range of 4-10 ng/ml). The presently disclosed methods, devices, combinations, kits, and systems may relate to detecting elevated sPSA in a sample from an individual.

The term “sample” should be interpreted to include, but not be limited to, bodily fluids (e.g., blood products including serum) and urine, as well as tissue samples (e.g., a prostate biopsy). The term sample should be interpreted to include serum samples having a serum prostate-specific antigen (sPSA) level of at least about 1, 2, 3, 4, 5, 6, 7, 8, 9, 10, 11, 12, 13, 14, or 15 ng/ml, or serum samples having a sPSA level within a range bounded by any of 1, 2, 3, 4, 5, 6, 7, 8, 9, 10, 11, 12, 13, 14, or 15 ng/ml (e.g., serum samples having a sPSA level within a range of 4-10 ng/ml).

The disclosed methods, devices, combinations, kits, and/or systems may include or utilize means and/or components for detecting mRNA of the genes DLX1, HOXC6, and/or KLK3. Means and components may include, but are not limited to one or more oligonucleotides that hybridize to mRNA of the genes DLX1, HOXC6, and/or KLK3 or that hybridize to reverse transcribed mRNA (e.g., cDNA) of the genes DLX1, HOXC6, and/or KLK3. Oligonucleotides may include a DNA primer form reverse transcribing mRNA of the genes DLX1, HOXC6, and/or KLK3. Oligonucleotides may include a pair of DNA primers for amplifying reverse transcribed mRNA (e.g., cDNA) of the genes DLX1, HOXC6, and/or KLK3. Means and components may include enzymes for reverse transcribing mRNA (e.g., a reverse transcriptase which optionally may be stable at temperatures >70° C.) and/or enzymes for amplifying reverse transcribed mRNA (i.e., DNA polymerases which optionally may be stable at temperature >70° C.).

Optionally, the disclosed methods, devices, combinations, kits, and/or systems may include or utilize means and/or components for detecting mRNA of the genes HOXC4, TDRD1, and/or KLK3. Means and components may include, but are not limited to one or more oligonucleotides that hybridize to mRNA of the genes HOXC4, TDRD1, and/or KLK3 or that hybridize to reverse transcribed mRNA (e.g., cDNA) of the genes HOXC4, TDRD1, and/or KLK3. Oligonucleotides may include a DNA primer form reverse transcribing mRNA of the genes HOXC4, TDRD1, and/or KLK3. Oligonucleotides may include a pair of DNA primers for amplifying reverse transcribed mRNA (e.g., cDNA) of the genes HOXC4, TDRD1, and/or KLK3. Means and components may include enzymes for reverse transcribing mRNA (e.g., a reverse transcriptase which optionally may be stable at temperatures >70° C.) and/or enzymes for amplifying reverse transcribed mRNA (i.e., DNA polymerases which optionally may be stable at temperature >70° C.).

The disclosed methods, devices, combinations, kits, and/or systems may utilize or include machines and/or components. Machines and.or components utilized by or including in the disclosed methods, devices, combinations, kits, and/or systems may include: machines and/or components for collecting a biological sample (e.g., machines and/or components for collecting urine); machines and/or components for obtaining, isolating, or preserving nucleic acid in a biological sample (e.g., a buffer or medium for obtaining, isolating, or preserving nucleic acid from urine); machines and/or components for reverse transcribing mRNA and/or preparing cDNA (e.g., nucleic acid reagents such as a primer, primer pair, or probe, and/or enzymes such as a reverse transcriptase including a thermostable reverse transcriptase); machines and/or components for amplifying cDNA (e.g., a thermocycling machine, primers or primer pairs which optionally are labelled, labeled probes, and/or a thermostable DNA polymerase); and machines and/or components for detecting amplified cDNA (e.g., machines for detecting a labelled amplicon such as fluorescent amplicon or for detecting a labeled probe).

The disclosed methods, devices, combinations, kits, and systems may include or utilize therapies or therapeutic agents for treating prostate cancer. Therapies for prostate cancer may include, but are not limited to performing surgery (e.g., surgery to remove cancerous prostate tissue), administering radiation therapy (e.g., radiation therapy directed at cancerous prostate tissue), administering radiopharmaceutical therapy (e.g., administering radiopharmaceuticals such as radio-labelled antibodies directed against cancerous prostate tissue), administering hormone therapy (e.g. administering anti-androgen therapy), administering chemotherapy, administering biologic therapy, administering bisphosphonate therapy, administering cryotherapy (e.g., cryotherapy directed against the cancerous prostate tissue), administering high-intensity focused ultrasound therapy (e.g., high-intensity focused ultrasound therapy directed against the cancerous prostate tissue), administering proton beam radiation therapy (e.g., proton beam radiation therapy directed against the cancerous prostate tissue), or a combination thereof.

### Examples

The following Examples are illustrative and should not be interpreted to limit the scope of the claimed subject matter

### Example 1—Initial Preclinical Discovery of Candidate Biomarkers

To identify markers for prostate cancer diagnosis and prediction of prognosis, the platform of Affymetrix GeneChips was used. Based on the robustness of this platform and the high resolution (many oligoprobes located in most exons of each gene) we decided to use the GeneChip Exon 1.0 ST array to determine the gene profiles of prostate tissue specimens that were collected at the Radboud University Nijmegen Medical Centre and Canisius Wilhemmina Hospital Nijmegen after a consent form approved by the institutional review board was signed by all participants.

Tissue specimens of patients with prostate cancer in the following groups were collected: normal prostate (NPr; n=8), Benign Prostatic Hyperplasia (BPH; n=12), Low grade prostate cancer (LG-PrCa; n=25), High grade prostate cancer (HG-PrCa; n=24), Castration resistant prostate cancer (CRPC; n=23) and prostate cancer metastases (PrCa Met; n=7).

Briefly, NPr tissue was obtained after radical or TURP from cancer free regions of these samples or from autopsy. BPH tissue was obtained from TURP or transvesical open prostatectomy (Hryntschak).LG-PrCa tissue was obtained from primary tumors with a Gleason Score ≤6, HG-PrCa tissue was obtained from primary tumors with a Gleason Score ≥7, CRPC tissue was obtained from patients that are progressive under endocrine therapy and who underwent a transurethral resection of the prostate (TURP) and PrCa Met tissue specimens were obtained from positive lymfnodes after LND or after autopsy. The tissues were snap frozen and cryostat sections were H.E. stained for classification by a pathologist.

Total RNA was extracted from tumor- and tumor-free areas using TRIzol (Invitrogen, Carlsbad, Calif., USA) following manufacturer's instructions. The total RNA was DNase treated and purified with the Qiagen RNeasy mini kit (Qiagen, Valencia, Calif., USA). Integrity of the RNA was checked by electrophoresis using the Agilent 2100 Bioanalyzer. Samples with RNA integrity number (RIN)>=6 were included.

Gene expression profiles were determined using the GeneChip Human Exon 1.0 Sense Target arrays (Affymetrix) according manufacturer's instructions. Gene-level and exon-level expression values were derived from the CEL file using the model-based Robust Multiarray Average algorithm as implemented in Partek® software (Partek Genomics Suite 6.6). P-values of differentially expressed genes between conditions were calculated using ANOVA analysis. For the identification of biomarkers the expression analysis of the different groups were compared: NP/BPH with LG- and HG-PCa, PCa-M+ with LG- and HG-PCa, CRPC with LG- and HG-PCa.

### Results and Conclusion

Using the GeneChip Exon 1.0 ST array we were able to identify an initial group of 47 interesting genes. These selected genes were further analysed and validated with RT-qPCR technique using Taqman Low Density Arrays (TLDA; Applied Biosystems) as will be further elucidated in example 2.

### Example 2—Preclinical Selection of Candidate Biomarkers

Further analysis of the 47 biomarkers was performed using Taqman® Low Density Arrays (TLDA; Applied Biosystems). Tissue and urine specimens were collected at the Radboud University Nijmegen Medical Centre and Canisius Wilhemmina Hospital Nijmegen. Tissue specimens of patients with prostate cancer in the following groups were collected: normal prostate (NPr; n=6), Benign Prostatic Hyperplasia (BPH; n=6), Low grade prostate cancer (LG-PrCa; n=14), High grade prostate cancer (HG-PrCa; n=14), Castration resistant prostate cancer (CRPC; n=14) and prostate cancer metastases (PrCa Met; n=8). Tissue selection and RNA extraction and purification were performed as described in example 1. Two ug DNase-treated total RNA was reverse transcribed using Superscript II Reverse Transcriptase (Invitrogen) according manufacturer's instructions.

For the validation not only prostate tissue specimens were used. To investigate whether the selected markers could successfully be detected in body fluids also normal bladder tissue specimens (n=2), peripheral blood lymphocytes (PBL, n=2) and urinary sediment specimens from patients which had PrCa in their biopsies (n=9) and 7 from patients with negative biopsies (n=7)) were included in the marker validation step. The background signal of the markers in normal bladder and urinary sediments from patients without prostate cancer should be low.

First voided urine samples were collected after digital rectal examination (DRE) from men scheduled for prostate cancer. After urine specimen collection, the urologist performed prostate biopsies according to a standard protocol. Prostate biopsies were evaluated and in case prostate cancer was present the Gleason score was determined. First voided urine after DRE (20-30 ml) was collected in a tube containing 2 ml 0.5M EDTA pH 8.0. All samples were immediately cooled to 4° C. and were mailed 10 in batches with cold packs to the laboratory.

The samples were processed within 48 h after the samples were acquired to guarantee good sample quality. Upon centrifugation at 4° C. and 1,800×g for 10 minutes, urinary sediments were obtained. These urinary sediments were washed twice with ice-cold buffered sodium chloride solution (at 4° C. and 1,800×g for 10 minutes), snap-frozen in liquid nitrogen, and stored at −70° C. Total RNA was extracted from these urinary sediments, using Trizol according to the manufacturers protocol. Two additional steps were added. Firstly, 2 μl glycogen (15 mg/ml) was added as a carrier (Ambion, Austin (Tex.), USA) before precipitation with isopropanol. Secondly, a second precipitation step with 3M sodium-acetate pH 5.2 and 100% ethanol was performed to discard traces of Trizol.

The RNA was DNase treated using amplification grade DNaseI (Invitrogen™ Breda, the Netherlands) according to the manufacturers protocol. Again glycogen was added as carrier and the RNA was precipitated with 3M sodium-acetate pH 5.2 and 100% ethanol for 2 hr at −20° C. The RNA was dissolved in 16.5 μl RNase-free water and 1 μg of total RNA was used for RNA amplification using the Ambion® WT Expression Kit (Ambion, Austin (Tex.), USA) according to the manufacturer's instructions. Two ug amplified RNA was reverse transcribed using Superscript II Reverse Transcriptase (Invitrogen) according manufacturer's instructions.

To determine gene expressions levels the cDNA generated from RNA extracted from both tissue specimens and urinary sediments was used as template in the TLDA's. After centrifugation of the 384-well TLDA cards for 1 minute at 280 g the cards were run in a 7900 HT Fast Real-Time PCR System (Applied Biosystems). Raw data were recorded with the Sequence detection System (SDS) software of the instruments analyzed with RQ documents and the RQ Manager Software for automated data analysis. Delta cycle threshold 30 (Ct) values were determined as the difference between the Ct of each test gene and the Ct of hypoxanthine phosphoribosyltransferase 1 (HPRT1) (endogenous control gene). Furthermore, gene expression values were calculated based on the comparative threshold cycle (Ct) method, in which a normal prostate RNA sample was designated as a calibrator to which the other samples were compared.

### Results

After analysis of the generated data, a list of 10 most promising biomarkers indicative for prostate cancer and the prognosis thereof was obtained.

### Example 3—Final Selection and Model Development of Candidate Biomarkers and Combinations Thereof in a Clinical Prospective Study

### Study Design

In a prospective multicenter study it was tested whether the ten in the pre-clinical biomarker discovery selected biomarkers or a combination of these markers could identify patients with PrCa_total or Sign-PrCa based on expression levels in urine samples. If so, these markers or a combination of markers could be used in an in vitro non-invasive method for diagnosing PrCa_total or Sign-PrCa.

Men who were scheduled for (initial or repeat) prostate biopsies, based on elevated sPSA levels, a family history of PCa or an abnormal DRE were included. First-catch urine after DRE was collected from 443 men. Prostate biopsies were performed and evaluated per hospital's standard procedure. In addition, one experienced genitourinary pathologist reviewed all biopsy Gleason scores independently, being blinded for the biomarker scores. Men were recruited at six urology clinics in the Netherlands (Radboud University Nijmegen Medical Centre, Nijmegen; Academic Medical Centre, Amsterdam; ZGT Hospital, Hengelo; Canisius Wilhelmina Hospital, Nijmegen; Scheper Hospital, Emmen; and St. Elisabeth Hospital, Tilburg). Exclusion criteria were: history of PCa, medical therapy known to affect sPSA levels, prostate biopsies within three months prior to enrolment, or invasive treatment for BPH within six months prior to enrolment. The respective independent ethics committees approved the study protocol and all included patients provided written informed consent. The biomarker discovery and the clinical validation study were both performed in accordance with the STARD (STAndards for Reporting of Diagnostic accuracy) criteria and REMARK (Reporting Recommendations for Tumor Marker Prognostic Studies) guidelines.

### Data Collection

Clinical pathological data were collected for each patient, including: age, sPSA, DRE and TRUS results, prostate volume, biopsy results (current and history), radiological results, clinical TNM stage (if diagnosed with PCa) and radical prostatectomy results (if applicable).

### Specimen Processing

First-catch urine specimens after DRE were processed using a validated standard operating procedure (SOP), total RNA was extracted from the urinary sediments, RNA was amplified and cDNA was generated as was described in example 2.

To determine the expression levels (copy numbers) for the selected biomarkers and for the genes KLK3, PCA3 and HPRT1 in these specimens, optimized real-time quantitative PCR assays were developed (according the MIQE guidelines). Fluorescence based real-time PCR assays with primers and hydrolysis probe were designed. PCR products were cloned into vectors and calibration curves with a wide linear dynamic range (10-1.000.000 copies) were made in order to calculate copy numbers.

Two μl of each cDNA sample was amplified in a 20 μl PCR reaction containing optimized amounts of forward primer and reverse primer, 2 pmol of hydrolysis probe and 1× Probes Master mix (Roche). The following amplification conditions were used: 95° C. for 10 minutes followed by 50 cycles at 95° C. for 10 seconds, 60° C. for 30 seconds and a final cooling step at 40° C. for 55 seconds (LightCycler LC480, Roche). The crossing point (Cp) values were determined using the Lightcycler 480 SW 1.5 software (Roche). The Cp values of the samples were converted to copy numbers by interpolation in the generated calibration curve. Samples that had HPRT1 mRNA <4000 copies were excluded for this study.

### Statistical Analyses

Statistical analyses were performed with SPSS® version 20.0. Two-sided P values of ≤0.05 were considered to indicate statistical significance. For a normal distribution expression data were log transformed and an univariate analysis was performed using forward logistic regression to determine whether the single biomarkers had significant predictive value (p<0.05) for diagnosis of PrCa_total and/or Sign-PrCa. The Odds Ratio's (O.R.) and corresponding 95% Confidence Intervals (CI) were determined.

To determine whether the markers had independent predictive value and had additional value to each other a multivariate analysis was performed using forward logistic regression. The best combinations of biomarkers for the prediction of PrCa or Sign-PrCa were identified.

To visualize the performance of the selected biomarkers in the absence of an arbitrary cut-off value, the data were summarized using a Receiver Operating Characteristic (ROC) curve. In a ROC curve, the true positive rate to detect prostate cancer (Sensitivity) is plotted in function of the false positive rate (i.e. positives in the no-PrCa group) (1-Specificity) for different cut-off points of a parameter. Each point on the ROC curve represents a sensitivity/specificity pair corresponding to a particular decision threshold. The area under the ROC curve is a measure of how well a parameter can distinguish between two groups, e.g. (PrCa_total versus no-PrCa). When the variable under study cannot distinguish between the two groups, i.e. in case there is no difference between the two distributions, the area will be equal to 0.5 (the ROC curve will coincide with the diagonal). A test with perfect discrimination (no overlap in the two distributions) has a ROC curve that passes through the upper left corner (100% sensitivity, 100% specificity). Therefore the closer the ROC curve is to the upper left corner, the higher the overall accuracy of the test.

### Results

In total, 443 patients were enrolled in this prospective multicenter study. Samples with HPRT1 mRNA <4000 copies were excluded (n=85). This resulted in 358 evaluable samples. In this cohort, 157 patients had prostate cancer in their biopsies. Of all the prostate cancers found (PrCa_total), 93 (59%) had a Gleason score >=7 and 64 (41%) had a Gleason score <=6. Furthermore, of all the prostate cancers found (PrCa_total), 118 (75%) could be classified as significant prostate cancer (Sign-PrCa), i.e. a Gleason Score of >=7 and/or a percentage biopsy positive cores >=33% and/or a clinical stage >=T2 (Epstein criteria)

Table 1A shows the univariate forward logistic regression data with the 10 biomarkers for diagnosis of PrCa. The P-values and Odds Ratios (O.R.) with corresponding 95% Confidence Intervals (CI) are presented. mRNA levels of HOXC4, HOXC6, DLX1, TDRD1, NKAIN1, PTPRT, CGREF1, GLYATL1 and PPFIA2 were significantly higher in patients with PrCa (PrCa_total) compared to patients without PrCa, whereas RRM2 was not.

TABLE 1A
Univariate forward logistic regression data
with the 10 biomarkers for diagnosis of PrCa.

95% C.I.

| | P-value | OR | Lower | Upper |
|---|---|---|---|---|
| LN_HOXC4 | 8.4E−06 | 1.35 | 1.18 | 1.54 |
| LN_HOXC6 | 1.7E−10 | 1.60 | 1.39 | 1.85 |
| LN_DLX1 | 4.7E−09 | 1.31 | 1.19 | 1.43 |
| LN_TDRD1 | 3.2E−07 | 1.21 | 1.12 | 1.30 |
| LN_NKAIN1 | 2.9E−04 | 1.21 | 1.09 | 1.34 |
| LN_PTPRT | 6.9E−06 | 1.19 | 1.10 | 1.28 |
| LN_RRM2 | 2.4E−01 | 1.11 | 0.94 | 1.31 |
| LN_CGREF1 | 3.0E−03 | 1.19 | 1.06 | 1.33 |
| LN_GLYATL1 | 2.0E−03 | 1.30 | 1.10 | 1.55 |
| LN_PPFIA2 | 2.5E−05 | 1.18 | 1.09 | 1.27 |

Table 1B shows the univariate forward logistic regression data with the 10 biomarkers for diagnosis of Sign-PrCa. mRNA levels of HOXC4, HOXC6, DLX1, TDRD1, NKAIN1, PTPRT, CGREF1, GLYATL1 and PPFIA2 were significantly higher in patients with Sign-PrCa compared to the rest (patients without PrCa or insignificant PrCa), whereas RRM2 was not.

TABLE 1B
Univariate forward logistic regression data
of 10 biomarkers for diagnosis of Sign-PrCa.

95% C.I.

| | P-value | OR | Lower | Upper |
|---|---|---|---|---|
| LN_HOXC4 | 6.7E−05 | 1.34 | 1.16 | 1.55 |
| LN_HOXC6 | 4.7E−10 | 1.65 | 1.41 | 1.94 |
| LN_DLX1 | 1.3E−11 | 1.36 | 1.24 | 1.48 |
| LN_TDRD1 | 1.0E−09 | 1.31 | 1.20 | 1.43 |
| LN_NKAIN1 | 6.1E−05 | 1.26 | 1.13 | 1.42 |
| LN_PTPRT | 4.8E−08 | 1.24 | 1.15 | 1.34 |
| LN_RRM2 | 5.9E−02 | 1.19 | 0.99 | 1.42 |
| LN_CGREF1 | 2.4E−04 | 1.28 | 1.12 | 1.47 |
| LN_GLYATL1 | 5.0E−03 | 1.30 | 1.08 | 1.56 |
| LN_PPFIA2 | 2.4E−05 | 1.20 | 1.10 | 1.30 |

Table 2A shows the multivariate analysis data with forward logistic regression for diagnosis of PrCa. Only DLX1 and HOXC6 had independent additional predictive value for diagnosing PrCa (P-value <0.05) and are stepwise added to the model.

TABLE 2A
Multivariate analysis data with forward logistic regression for diagnosis of PrCa.

| Variables in the Equation | | | | | | | | |
|---|---|---|---|---|---|---|---|---|
| | | | | 95% C.I. | | Variables not in the Equation | | |
| | | P-value | O.R. | Lower | Upper | | | P-value |
| Step 1 | LN_HOXC6 | <0.001 | 1.61 | 1.39 | 1.86 | Step 2 | LN_HOXC4 | 0.40 |
| | Constant | <0.001 | 0.04 | | | | LN_TDRD1 | 0.20 |
| Step 2 | LN_HOXC6 | <0.001 | 1.47 | 1.26 | 1.71 | | LN_PPFIA | 0.40 |
| | LN_DLX1 | <0.001 | 1.21 | 1.105 | 1.34 | | LN_GLYATL1 | 0.33 |
| | Constant | <0.001 | 0.05 | | | | LN_RRM2 | 0.20 |
| | | | | | | | LN_CGREF1 | 0.06 |
| | | | | | | | LN_NKAIN1 | 0.60 |
| | | | | | | | LN_PTPRT | 0.72 |
| | | | | | | | LN_RRM2 | 0.20 |

Table 2B shows the multivariate analysis data with forward logistic regression for diagnosis of Sign-PrCa. Only DLX1, HOXC6 and TDRD1 had independent additional predictive value for diagnosing Sign-PrCa (P-value <0.05) and are stepwise added to the model.

TABLE 2B
multivariate analysis data with forward logistic regression for diagnosis of Sign-PrCa.

| Variables in the Equation | | | | | | | | |
|---|---|---|---|---|---|---|---|---|
| | | | | 95% C.I. | | Variables not in the Equation | | |
| | | P-value | OR | Lower | Upper | | | P-value |
| Step 1 | LN_DLX1 | <0.001 | 1.37 | 1.25 | 1.49 | Step 3 | LN_HOXC4 | 0.75 |
| | Constant | <0.001 | 0.27 | | | | LN_GLYATL1 | 0.18 |
| Step 2 | LN_HOXC6 | <0.001 | 1.43 | 1.21 | 1.69 | | LN_NKAIN1 | 0.44 |
| | LN_DLX1 | <0.001 | 1.26 | 1.14 | 1.40 | | LN_PPFIA2 | 0.72 |
| | Constant | <0.001 | 0.03 | | | | LN_PTPRT | 0.49 |
| Step 3 | LN_HOXC6 | <0.001 | 1.36 | 1.15 | 1.62 | | LN_RRM2 | 0.15 |
| | LN_DLX1 | 0.001 | 1.20 | 1.08 | 1.34 | | LN_CGREF1 | 0.10 |
| | LN_TDRD1 | 0.013 | 1.14 | 1.03 | 1.26 | | | |
| | Constant | <0.001 | 0.03 | | | | | |

HOXC4 and HOXC6 are transcribed from the same transcriptional unit. The expression patterns of both genes show high correlation and both are significant diagnostic and prognostic biomarkers. In the multivariate logistic regression with the 10 biomarkers and with both genes included HOXC4 is not an independent predictor. HOXC6 performs better for diagnosing PrCa_total and Sign-PrCa than HOXC4 however, the differences are very small and these markers can be interexchanged in many cases. When a multivariate analysis is performed without either HOXC4 or HOXC6, of all other 8 biomarkers again only DLX1 and/or TDRD1 have independent additional value and are added to the model for diagnosing PrCa_total and Sign-PrCa. Therefore it was decided to perform further detailed data analysis for the four genes HOXC4, HOXC6, DLX1 and TDRD1 and combinations thereof.

In Tables 3A and 3B, the copy numbers, copy number ranges and fold-changes between the groups of the individual genes of interest are shown for no PrCa, and PrCa_total (diagnosis) and for Sign-PrCa and the rest (prognosis) for the cohort of 358 urinary sediments.

Table 3A, B: copy numbers, ranges and fold-changes between the groups of the individual genes

### A.

| | | | | | Fold |
|---|---|---|---|---|---|
| | PrCa | | no PrCa | | Change |
| | n = 157 | | n = 201 | | PrCa/ |
| | copies | Range | copies | Range | no PrCa |
| HOXC4 | 25288 | (1-355000) | 8888 | (1-116000) | 2.9 |
| HOXC6 | 5433 | (1-193000) | 729 | (1-8970) | 7.5 |
| TDRD1 | 17629 | (1-727000) | 619 | (1-56400) | 28.5 |
| DLX1 | 1510 | (1-66700) | 42 | (1-3690) | 36.2 |

### B.

| | | | | | Fold |
|---|---|---|---|---|---|
| | Sign PrCa | | Rest* | | Change |
| | n = 118 | | n = 240 | | Sign PrCa/ |
| | copies | Range | copies | Range | Rest |
| HOXC4 | 29843 | (1-355000) | 9313 | (1-116000) | 3.2 |
| HOXC6 | 6873 | (1-193000) | 785 | (1-8970) | 8.8 |
| TDRD1 | 23363 | (1-727000) | 587 | (1-56400) | 39.8 |
| DLX1 | 1996 | (1-66700) | 42 | (1-3690) | 47.7 |

Rest* = neg. biopsy and insign PrCa (GS < 7, biopsy pos. cores <33% and < T2 stage)

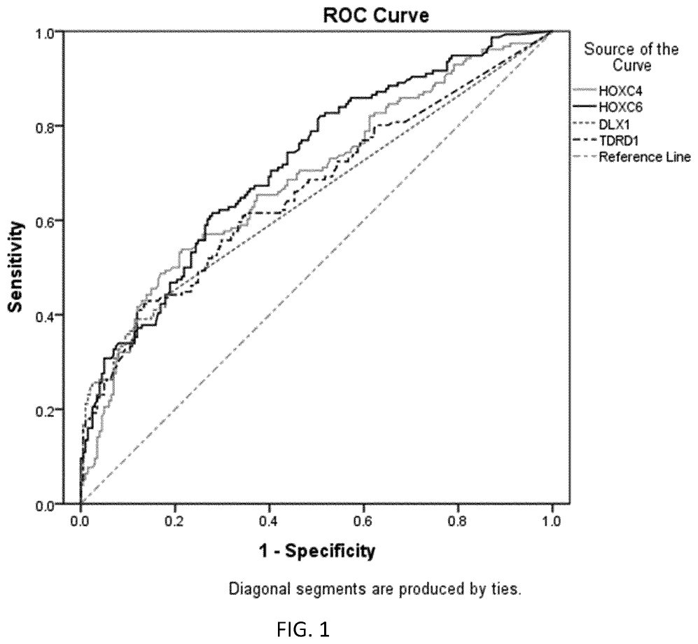

To visualize the performance of the four selected biomarkers to discriminate PrCa_total from no-PrCa and to discriminate Sign-PrCa from the rest (i.e. negative biopsy and insignificant PrCa) the data were summarized using a Receiver Operating Characteristic (ROC) curve. The ROC curves were made for the four single biomarkers and for the combinations of these biomarkers. In a Receiver under Operation (ROC)-curve, the diagnostic potential of HOXC4, HOXC6, DLX1 and TDRD1 expression in the cohort of 358 urinary sediments to discriminate no PrCa from PrCa_total is visualized in FIG. 1. The area under the curve (AUC) for HOXC4 is 0.69 (95% CI: 0.63-0.74), for HOXC6 is 0.72 (95% CI: 0.67-0.77), for DLX1 is 0.65 (95% CI: 0.59-0.71) and for TDRD1 is 0.66 (95% CI: 0.60-0.72).

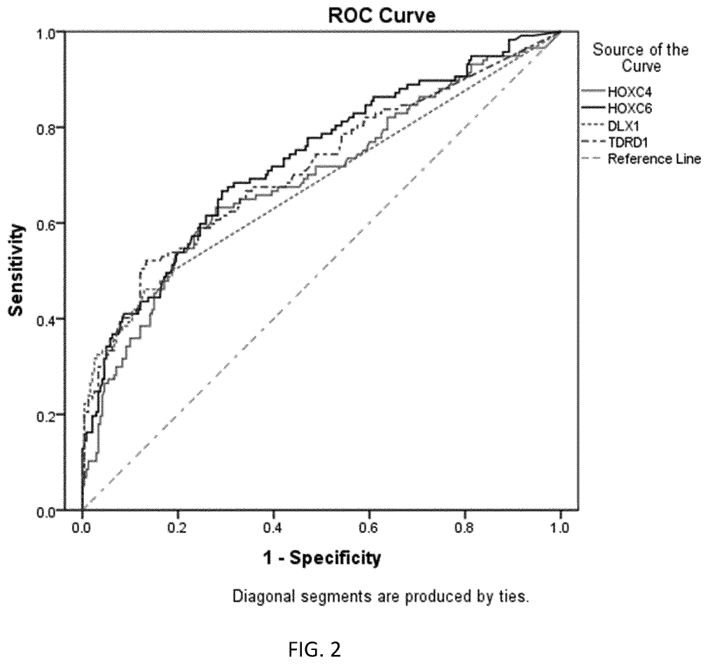

In a Receiver under Operation (ROC)-curve, the prognostic potential of HOXC4, HOXC6, DLX1 and TDRD1 expression in urinary sediments to discriminate Sign-PrCa from the rest (i.e. insignificant PrCa and negative biopsies) is visualized in FIG. 2. The area under the curve (AUC) for HOXC4 is 0.69 (95% CI: 0.63-0.75), for HOXC6 is 0.73 (95% CI:0.67-0.79), for DLX1 is 0.68 (95% CI: 0.62-0.74) and for TDRD1 is 0.71 (95% CI: 0.65-0.77).

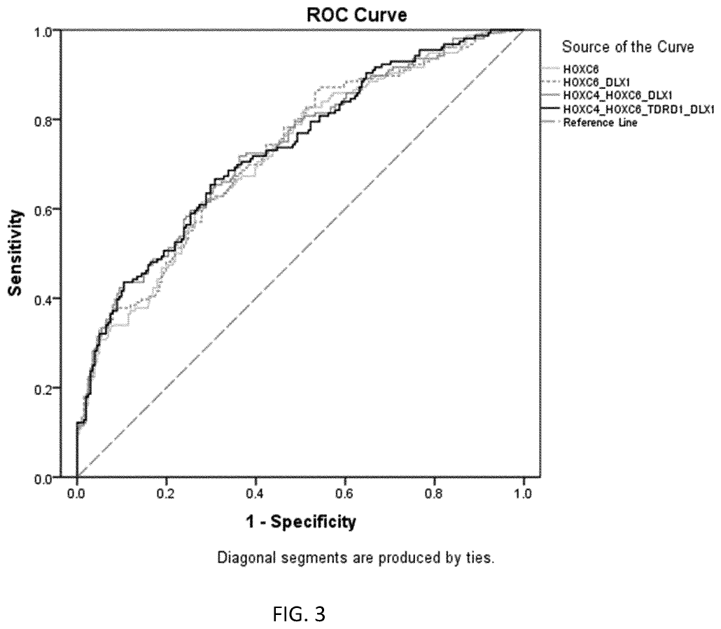

The diagnostic potential of combinations of HOXC4, HOXC6, DLX1 and TDRD1 for the expression in the cohort of 358 urinary sediments to discriminate no PrCa from PrCa_total is visualized in a Receiver under Operation (ROC)-curve in FIG. 3. The area under the curve (AUC) for HOXC6 expression is 0.719 (95% CI: 0.67-0.77), for the combination of HOXC6 with DLX1 is 0.726 (95% CI:0.67-0.78), for the combination of HOXC6, DLX1 and HOXC4 expression is 0.733 (95% CI: 0.68-0.79) and for the combination of HOXC6, DLX1, HOXC4 and TDRD1 is 0.732 (95% CI: 0.68-0.78).

To discriminate no PrCa from PrCa_total (diagnosis) in urinary sediments, at a specificities of 70%, 80% and 90% cut-off values were extrapolated from the ROC-curves and the sensitivity, positive predictive value (PPV), negative predictive value (NPV) was calculated for HOXC6 and for the combinations with HOXC4, DLX1 and TDRD1. Furthermore the number of patients with prostate cancer detected by the markers was determined as well. The results are summarized in Table 4A.

TABLE 4A
Discrimination of no PrCa from PrCa total (diagnosis) in urinary sediments

| | | Spec. 70% | | | | Spec. 80% | | | | Spec. 90% | | | |
|---|---|---|---|---|---|---|---|---|---|---|---|---|---|
| | AUC | Sens. | PPV | NPV | PCa | Sens. | PPV | NPV | PCa | Sens. | PPV | NPV | PCa |
| HOXC6 | 0.719 | 62 | 62 | 70 | 97 | 46 | 65 | 66 | 73 | 34 | 73 | 64 | 53 |
| HOXC6/DLX1 | 0.726 | 62 | 62 | 70 | 97 | 48 | 65 | 66 | 75 | 38 | 75 | 65 | 59 |
| HOXC6/HOXC4/DLX1 | 0.733 | 64 | 63 | 71 | 100 | 50 | 66 | 67 | 78 | 42 | 77 | 67 | 66 |
| HOXC6/HOXC4/DLX1/TDRP1 | 0.732 | 65 | 63 | 72 | 102 | 51 | 66 | 68 | 79 | 41 | 76 | 66 | 64 |
| HOXC4/TDRD1/DLX1 | 0.722 | 63 | 62 | 71 | 98 | 53 | 67 | 69 | 83 | 39 | 75 | 66 | 61 |
| serum PSA | 0.658 | 55 | 59 | 67 | 87 | 41 | 62 | 64 | 65 | 29 | 70 | 62 | 46 |
| mPCA3 | 0.716 | 62 | 62 | 70 | 97 | 44 | 63 | 65 | 69 | 26 | 67 | 61 | 41 |

PCa = number of detected cancer patients

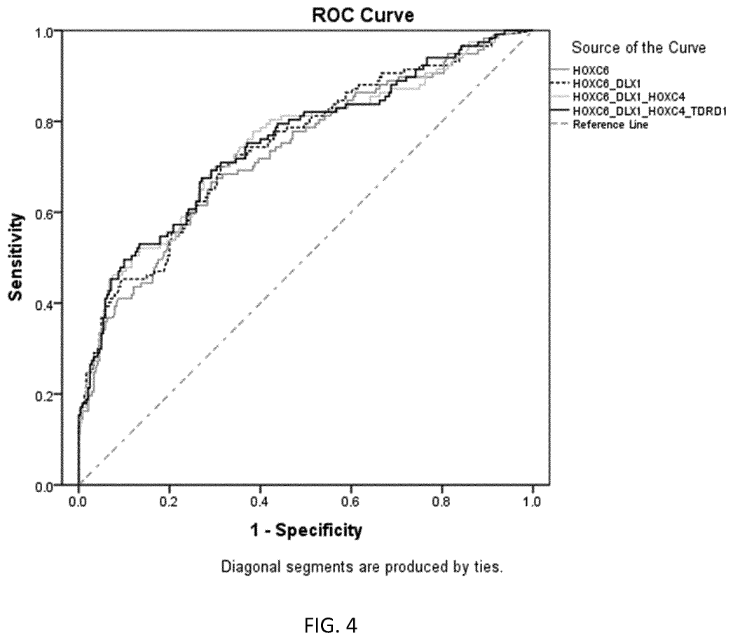

The prognostic potential of combinations of HOXC4, HOXC6, DLX1 and TDRD1 for the expression in the cohort of 358 urinary sediments to discriminate Sign-PrCa from the rest (insignificant PrCa and negative biopsies) are visualized in a Receiver under Operation (ROC)-curve in FIG. 4. The area under the curve (AUC) for HOXC6 expression is 0.731 (95% CI: 0.67-0.79), for the combination of HOXC6 with DLX1 is 0.745 (95% CI:0.69-0.80), for the combination of HOXC6, DLX1 and HOXC4 is 0.750 (95% CI: 0.69-0.81) and for the combination of HOXC6, DLX1, HOXC4 and TDRD1 expression is 0.753 (95% CI: 0.70-0.81).

To discriminate Sign-PrCa from the rest (prognosis) in urinary sediments, at a specificities of 70%, 80% and 90% cut-off values were extrapolated from the ROC-curves and the sensitivity, positive predictive value (PPV), negative predictive value (NPV) were calculated for HOXC6 and for the combinations with HOXC4, DLX1 and TDRD1. Furthermore the number of significant prostate cancers detected was determined as well. The results are summarized in Table 4B.

TABLE 4B
Discrimination of Sign-PrCa from the rest (prognosis) in urinary sediments

| | | Spec. 70% | | | | Spec. 80% | | | | Spec. 90% | | | |
|---|---|---|---|---|---|---|---|---|---|---|---|---|---|
| | AUC | Sens. | PPV | NPV | PCa | Sens. | PPV | NPV | PCa | Sens. | PPV | NPV | PCa |
| HOXC6 | 0.731 | 66 | 52 | 81 | 78 | 53 | 57 | 78 | 63 | 41 | 67 | 76 | 48 |
| HOXC6/D1X1 | 0.745 | 65 | 52 | 80 | 77 | 53 | 57 | 78 | 63 | 45 | 69 | 77 | 53 |
| HOXC6/HOXC4/DLX1 | 0.750 | 70 | 54 | 83 | 83 | 54 | 57 | 78 | 64 | 47 | 70 | 77 | 55 |
| HOXC6/HOXC4/DLX1/TDRD1 | 0.753 | 69 | 53 | 82 | 81 | 56 | 58 | 79 | 65 | 50 | 71 | 79 | 58 |
| HOXC4/TDRD1/DLX1 | 0.749 | 70 | 53 | 83 | 82 | 58 | 59 | 80 | 68 | 43 | 68 | 76 | 50 |
| serum PSA | 0.693 | 57 | 49 | 77 | 68 | 49 | 55 | 76 | 58 | 36 | 63 | 74 | 43 |
| mPCA3 | 0.706 | 59 | 49 | 77 | 69 | 44 | 52 | 74 | 52 | 25 | 55 | 71 | 29 |

PCa = number of detected cancer patients

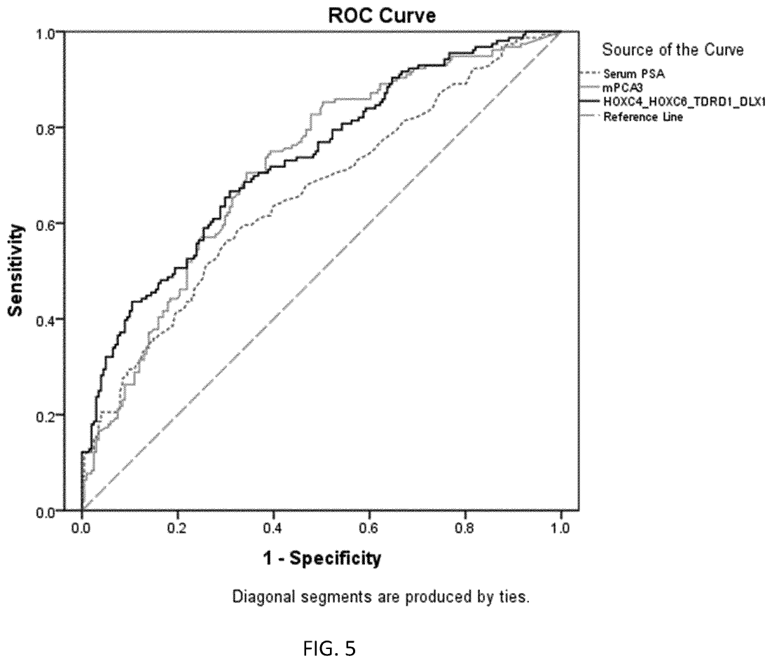

In a Receiver under Operation (ROC)-curve, the diagnostic potential of the combination of HOXC6, DLX1, HOXC4 and TDRD1 expression and the expression of PCA3 in urinary sediments and the serum PSA value to discriminate no PrCa from PrCa_total is visualized in FIG. 5. The area under curve (AUC) for the combination of HOXC6, DLX1, HOXC4 and TDRD1 expression is 0.732 (95% CI: 0.68-0.78), for the expression of PCA3 is 0,716 (95% CI: 0.66-0.77) and for serum PSA is 0,658 (95% CI: 0.60-0.72).

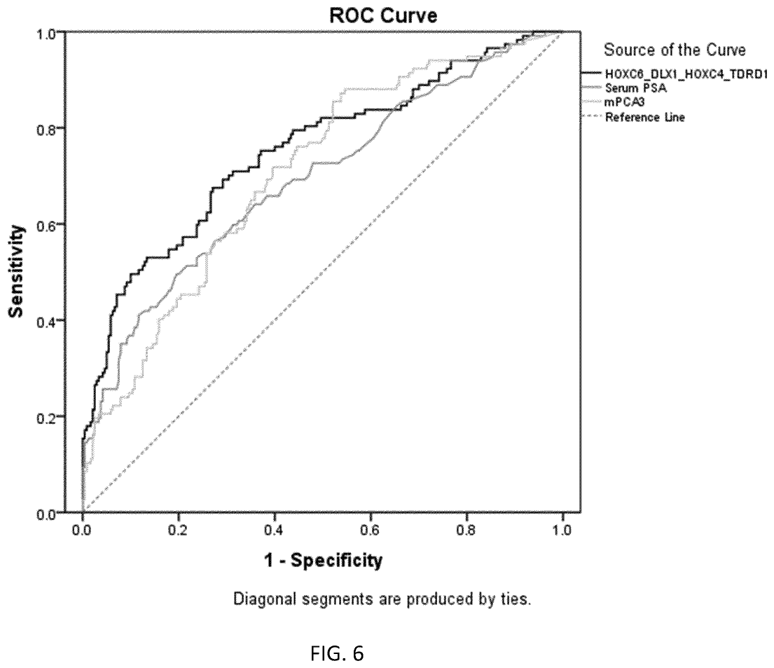

In a Receiver under Operation (ROC)-curve, the prognostic potential of the combination of HOXC6 with DLX1, HOXC4 and TDRD1 expression, the expression of PCA3 in urinary sediments and the serum PSA value to discriminate Sign-PrCa from the rest (insignificant PrCa and negative biopsies) is visualized in FIG. 6. The area under curve (AUC) for the combination of HOXC6, DLX1, HOXC4 and TDRD1 expression is 0.753 (95% CI: 0.70-0.81), for the expression of PCA3 is 0,706 (95% CI: 0.65-0.76) and for serum PSA is 0,693 (95% CI: 0.63-0.75).

### Discussion

The present example shows that the individual selected genes (HOXC4, HOXC6, DLX1 and TDRD1) each show overexpression in prostate cancer versus no-prostate cancer (diagnosis) and overexpression in Sign-PrCa versus the rest (prognosis). Especially TDRD1 and DLX1 show in both situations a high fold change difference between the groups.

Based on the AUC of the ROC curves for the single biomarkers, HOXC6 shows the best results for diagnosing prostate cancer (PrCa_total) (FIG. 1). When combined with DLX1, HOXC4 and or TDRD1 the overall diagnostic performance is improved. This does not mean that this combination is the best under all conditions. Depending on where the biomarkers will be used for (screening, reduction of biopsies) a high specificity or a high sensitivity is desired. If a higher sensitivity is desired, e.g. at a lower specificity of 80%, the combination of HOXC4, DLX1 and TDRD1 performs best (see Table 4A).

At a specificity of 70%, HOXC6 detects 97 of the prostate cancers (sensitivity of 62%), when combined with HOXC4, DLX1 and TDRD1 102 prostate cancers are detected (sensitivity of 65%). If a higher specificity is required, e.g. at 80%, the combination of HOXC4, DLX1 and TDRD1 performs best; 83 cancers are detected (sensitivity 53%) and this is a significant improvement compared to the number of cancers detected by serum PSA (65) and mPCA3 (69). These tests have a sensitivity of only 41% and 44% respectively at a specificity of 80% (Table 4A).

For the detection of significant prostate cancer (prognosis), from the 4 single biomarkers HOXC6 shows the best results based on the AUC of the ROC curves (FIG. 6).

When combined with DLX1, HOXC4 and or TDRD1 the prognostic performance is improved, especially at a high specificity.

At a specificity of 90%, HOXC6 detects 48 of the significant prostate cancers (sensitivity of 41%), when combined with HOXC4, DLX1 and TDRD1 58 significant prostate cancers are detected (sensitivity of 50%). This is a significant improvement compared to the number of cancers detected by serum PSA (43) and mPCA3 (29). These tests have a sensitivity of only 36% and 25% respectively at a specificity of 90% (Table 4B).

For a urologist the probability of having significant prostate cancer should be as high as possible. Therefore the specificity and PPV are very important for treatment decision making. At a high specificity of 90%, the PPV of the four gene panel is 71%. This indicates that a man with a positive test for the four gene panel has a probability of 71% for having significant prostate cancer. This is significantly higher than the PPV for serum PSA (63%) and mPCA3 (55%).

### CONCLUSION

As demonstrated above, the present molecular markers, or biomarkers, for prostate cancer provide, in combination, methods and means allowing discrimination between prostate cancer and no-prostate cancer and allowing detecting significant prostate cancer from the rest (insignificant PrCa and negative biopsies) with improved sensitivity, specificity, PPV and NPV, especially when compared with presently available biomarkers such as the serum PSA and mPCA3.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

What is claimed is:

1. Method for in vitro establishing prostate cancer (PrCa) in a sample originating from a human individual suspected of or suffering from prostate cancer comprising:
a) determining expression levels of DLX1 and HOXC6;
b) establishing up-regulation of the expression levels of DLX1 and HOXC6 as compared to expression levels of DLX1 and HOXC6 in a sample originating from an individual not suffering from prostate cancer or as compared to a reference value indicative of a non-disease expression level;
c) establishing the presence or absence of prostate cancer (PrCa) based on the up-regulation of the expression levels of DLX1 and HOXC6 in said sample; and
d) administering treatment for prostate cancer to the individual if the presence of prostate cancer is established.

2. Method according to claim 1, wherein step (a) further comprises determining the expression level of HOXC4; wherein step (b) further comprises establishing up-regulation of the expression level of HOXC4 as compared to expression level of HOXC4 in a sample originating from an individual not suffering from prostate cancer or as compared to a reference value indicative of a non-disease expression level; and wherein step (c) further comprises establishing the presence or absence of prostate cancer (PrCa) based on the up-regulation of the expression levels of DLX1, HOXC6 and HOXC4 in said sample.

3. Method according to claim 1, wherein step (a) further comprises determining the expression level of TDRD1; wherein step (b) further comprises establishing up-regulation of the expression level of TDRD1 as compared to expression level of TDRD1 in a sample originating from an individual not suffering from prostate cancer or as compared to a reference value indicative of a non-disease expression level; and wherein step (c) further comprises establishing the presence or absence of prostate cancer (PrCa) based on the up-regulation of the expression levels of DLX1, HOXC6 and TDRD1 in said sample.

4. Method according to claim 1, wherein step (a) further comprises determining the expression levels of HOXC4 and TDRD1; wherein step (b) further comprises establishing up-regulation of the expression levels of HOXC4 and TDRD1 as compared to expression levels of HOXC4 and TDRD1 in a sample originating from an individual not suffering from prostate cancer or as compared to a reference value indicative of a non-disease expression level; and wherein step (c) further comprises establishing the presence or absence of prostate cancer (PrCa) based on the up-regulation of the expression levels of DLX1, HOXC6, HOXC4 and TDRD1 in said sample.

5. Method according to claim 1, wherein establishing prostate cancer (PrCa) comprises one ore more of the following:
(a) establishing high grade (Gleason score >=7) or low grade (Gleason score <7) prostate cancer;
(b) establishing a percentage of positive cores of >=33%; and
(c) establishing a clinical stage of >=T2 according to the Epstein criteria.

6. Method according to claim 1, wherein determining expression levels comprises determining mRNA expression levels.

7. Method according to claim 1, wherein said sample is a sample selected from the group consisting of urine, urine derived, prostatic fluid, prostatic fluid derived, ejaculate and ejaculate derived.

8. Method of claim 1, comprising determining mRNA expression levels of the genes DLX1 and HOXC6 in a urine sample from an individual having a serum prostate-specific antigen (sPSA) level of 4-10 ng/ml.

9. Method of claim 1, comprising determining mRNA expression levels of the genes DLX1 and HOXC6 and one ore more of the genes HOXC4 and TDRD1 in a urine sample from an individual having a serum prostate-specific antigen (sPSA) level of 4-10 ng/ml.

10. Method according to claim 1, wherein the treatment for prostate cancer comprises performing surgery, administering radiation therapy, administering radiopharmaceutical therapy, administering hormone therapy, administering chemotherapy, administering biologic therapy, administering bisphosphonate therapy, administering cryotherapy, administering high-intensity focused ultrasound therapy, administering proton beam radiation therapy, or a combination thereof.

11. Method comprising administering treatment for prostate cancer to an individual exhibiting up-regulation of expression levels of DLX1 and HOXC6 in a urine sample.

12. Method of claim 11, wherein the individual further is exhibiting up-regulation of expression levels of one or more of HOXC4 and TDRD1 in the urine sample.

13. Method of claim 11, wherein the patient has a serum prostate-specific antigen (sPSA) level of 4-10 ng/ml.

14. Method comprising:
(a) requesting a test providing results of an analysis to determine the mRNA expression levels of the genes DLX1 and HOXC6 in a urine sample; and
(b) administering treatment for prostate cancer based on the results of the test.

15. Method of claim 14, wherein treatment is administering if the results indicate elevated mRNA expression levels of the genes DLX1 and HOXC6 in the urine sample, for example relative to an internal control such as KLK3 mRNA or relative to an external control such as mRNA expression levels of the genes DLX1 and HOXC6 in an individual not having prostate cancer.

16. Method of claim 14, wherein the test further provides results of an analysis to determine the mRNA expression levels of the genes HOXC4 or TDRD1 in the urine sample.

17. Method according to claim 14, further comprising one or more of the following:
(a) requesting a test providing results of an analysis to determine the concentration of prostate-specific antigen (sPSA) in a serum sample from the individual and administering treatment for prostate cancer based on the results of the test; and
(b) requesting a test providing results of an analysis to determine the Gleason score of a prostate biopsy of the individual and administering treatment for prostate cancer based on the results of the test.

18. A system comprising:
(a) components for obtaining RNA from a urine sample; and
(b) components for detecting mRNA of the genes DLX1 and HOXC6 in the obtained RNA.

19. The system of claim 18, wherein the components for detecting mRNA of the genes DLX1 and HOXC6 comprise one or more of the following:
(a) an oligonucleotide that hybridizes to mRNA of the genes DLX1 and HOXC6; and
(b) a pair of oligonucleotides for amplifying reverse transcribed mRNA of the genes DLX1 and HOXC6.

20. The system of claim 18, further comprising: (c) components for detecting mRNA of the genes HOXC4 or TDRD1 in the obtained RNA.

21. The system of claim 18, further comprising: (d) components for obtaining a urine sample.

22. The system of claim 18, further comprising a therapeutic agent for treating prostate cancer selected from the group consisting of radiopharmaceutical therapeutic agent, a hormonal therapeutic agent, a chemotherapeutic agent, a biological therapeutic agent, a bisphosphonate therapeutic agent, or a combination thereof.

23. Kit of parts for in vitro establishing prostate cancer in a sample originating from human individual suspected of suffering from prostate cancer comprising:
expression level analysis means for determining the expression levels of HOXC6 and DLX1.

24. Kit of parts according to claim 23 further comprising expression level analysis means for determining the expression level of one or more of HOXC4 and TDRD1.
